(19) United States
(12) Patent Application Publication
(10) Pub. No.: US 2005/0191302 A1
(43) Pub. Date: Sep. 1, 2005

(54) Treatment for liver disease

(75) Inventors: Michael James Paul Arthur, Southampton (GB); Derek Austin Mann, Southampton (GB); John Peter Iredale, Southampton (GB); Christopher Benyon, Southampton (GB); Frank Murphy, Southampton (GB); Fiona Oakley, Southampton (GB); Richard Ruddell, Southampton (GB); Matthew Christopher Wright, Aberdeen (GB)

Correspondence Address: NIXON & VANDERHYE, PC, 901 NORTH GLEBE ROAD, 11TH FLOOR, ARLINGTON, VA, 22203, US

(73) Assignee: UNIVERSITY OF SOUTHAMPTON, Southampton (GB)

(21) Appl. No.: 10/650,074

(22) Filed: Aug. 28, 2003

Related U.S. Application Data
(60) Provisional application No. 60/406,669, filed on Aug. 29, 2002.

(51) Int. Cl. A61K 39/395

(52) U.S. Cl. 424/145.1; 424/146.1

(57) ABSTRACT

The present invention is based on the finding that the artificial induction of hepatic stellate cell (HSC) apoptosis in vivo can promote the resolution of liver fibrosis. Thus, the present invention provides methods for treating liver disease in a subject involving administration of an inducer of apoptosis which is capable of selectively inducing hepatic stellate cell apoptosis in the liver of the subject or of an agent which is capable of giving rise to such an inducer in the subject. In addition, the invention provides methods for treating liver fibrosis in a subject comprising the selective delivery of an inducer of apoptosis specifically to the hepatic stellate cells of the subject or of an agent which is capable of giving rise to an inducer of hepatic stellate cell apoptosis.

### FIELD OF THE INVENTION

The present invention relates to methods for treating liver disease and in particular to methods for promoting the resolution of liver fibrosis.

### BACKGROUND OF THE INVENTION

Liver fibrosis is characterized by an accumulation of extracellular matrix proteins. Although the liver has a certain capacity for the breakdown of the matrix deposited in fibrosis, and hence for the resolution of fibrosis, in some cases fibrosis is not successfully resolved and instead progressively increases. This results in increasing impairment of liver function with the fibrotic material disturbing the overall organization of the liver, altering blood flow and causing the destruction of liver cells.

Liver fibrosis may progress to cirrhosis with the liver taking on a nodular structure with islands of healthy or regenerating liver tissue surrounded by regions of fibrotic and necrotic material. As liver fibrosis progresses the affected individual will experience severe illness, often being repeatedly hospitalized, and ultimately liver failure and death may occur. Liver disease is one of the most frequent causes of death in the 30 to 60 age range and in many cases the only effective treatment at present is a liver transplant.

Liver fibrosis can have a number of causes and is a common response to chronic hepatic damage. It may be mediated by a variety of mechanisms including: xenobiotic damage (for example it can be caused by consumption of alcohol in excessive amounts over prolonged periods or be due to certain drugs); viral infection (for example Hepatitis B or C infection); and certain genetic diseases (such as, for example, hepatic hemochromatosis—Friedman et al., N. Engl. J. Med., (1993) 328:1828-1835).

One of the factors which may decide whether the fibrosis is transient or progressive is the underlying cause of the fibrosis and whether or not there is only transient exposure to the causative agent or exposure is prolonged. In cases, for example, where there is continued exposure to the causative agent, this may mean that the liver never effectively gets a chance to resolve the fibrosis. Although there may be periods of some resolution, overall the trend will be a progressive buildup of fibrotic material.

Hepatic stellate cells (HSCs) are known to play a central role in liver fibrosis (Friedman et al., J. Biol. Chem., (2000) 275:2247-2250, Alcolado et al., Clin. Sci., (1997) 92:103-112 and Iredale et al., J. Clin. Invest., (1998) 102:538-549). Hepatic stellate cells are localized in the liver within the space of Disse and function to store retinoids. Interestingly, hepatic stellate cells are capable of synthesizing both factors capable of promoting fibrosis, but also factors thought to promote the resolution of fibrosis.

In response to liver damage, hepatic stellate cells “activate” to a myofibroblast like (α-smooth muscle actin-expressing) phenotype. Current evidence indicates that activated hepatic stellate cells synthesize the majority of extracellular matrix protein deposited in liver fibrosis (Milani et al., Hepatology (1989) 10:84-92). However, stellate cells can also release an array of matrix metalloproteases (MMPs). Some of these MMPs can degrade the matrix proteins laid down in fibrosis and hence promote resolution. In addition, hepatic stellate cells can also release TIMPs (tissue inhibitors of matrix metalloproteases) which are capable of inhibiting specific MMPs, involved in matrix degradation, preventing the breakdown of fibrotic material and hence promoting the overall buildup of fibrosis. It is thought that the amount and type of factors released by hepatic stellate cells, together with the interplay between these factors helps to determine whether there is a net prgession or regression of liver fibrosis.

### SUMMARY OF THE INVENTION

The present invention is based on the finding that the selective induction of hepatic stellate cell apoptosis in the liver can promote or enhance the resolution of liver disease and in particular of liver fibrosis in a subject. It is also possible that the induction of hepatic stellate cell apoptosis may prevent the buildup of fibrotic material. Thus by specifically inducing hepatic stellate cell apoptosis liver disease can be treated.

Accordingly, the present invention provides a method of treating liver disease in a subject, the method comprising administering to said subject an effective amount of an inducer of hepatic stellate cell apoptosis, or of an agent capable of giving rise to an inducer of hepatic stellate cell apoptosis, wherein said inducer or agent:
(a) is selectively delivered to hepatic stellate cells in the liver of the subject;
(b) selectively induces, or gives rise to a selective inducer, of hepatic stellate cell apoptosis in the liver of the subject; and/or
(c) generates an inducer of apoptosis specifically in hepatic stellate cells.

The invention also provides a kit comprising:
a selective inducer of hepatic stellate cell apoptosis or an agent capable of giving rise to a selective inducer of hepatic stellate cell apoptosis in vivo; and
instructions describing how to administer the inducer or agent to a subject suffering from liver disease.

The invention further provides a kit comprising:
an inducer of hepatic stellate cell apoptosis or an agent capable of giving rise to an inducer of hepatic stellate cell apoptosis in vivo;
instructions describing how to selectively deliver the inducer or agent to the hepatic stellate cells of a subject suffering from liver disease.

In an especially preferred embodiment of the invention the inducer of hepatic stellate cell apoptosis employed is an antagonist of a 5HT₂ receptor. In another especially preferred embodiment of the invention the inducer is sulfasalazine or a derivarive thereof capable of inducing hepatic stellate cell apoptosis.

### BRIEF DESCRIPTION OF THE FIGURES

FIG. 1. Structure of gliotoxin and mt-glio (bis-dethio-bis(methylthio)-gliotoxin.

FIG. 2. Effect of gliotoxin on caspase 3 activity and DNA integrity in rat hepatic stellate cells. Panel A shows the level of caspase 3 activity in rat hepatic stellate cells treated with DMSO (control), gliotoxin, Z-VAD-FMK, chlorpromazine, or with gliotoxin and Z-VAD-FMK together. The asterisked bar (*) indicates significantly different (P>95%) activity versus control cells using the Student t test (two-tailed). Panel B shows the results of FACS analysis of rat hepatic stellate cells treated with either DMSO (clear) or gliotoxin (shaded) and stained with propidium iodide.

FIG. 3. Comparison of cell death in rat hepatocytes and hepatic stellate cells in response to a variety of stimuli. Panel A compares the percentage of rat hepatocytes (open circles) and hepatic stellate cells (shaded boxes) remaining attached to the culture vessel at a given gliotoxin concentration. Panel B shows the percentage of viable rat hepatocytes as judged by attachment (open bars) and trypan blue staining to assess membrane integrity (shaded bars) following treatment with DMSO, gliotoxin, chlorpromazine, TNF-α with cycloheximide, or methapyrilene.

FIG. 4. Panel A shows an example of an apoptotic hepatic stellate cell (arrow) induced to undergo apoptosis by cycloheximide exposure and identified in situ by acridine orange staining. A normal cell lies adjacent to the apoptotic body. Panel B shows the proportion of apoptotic cells (expressed as a percentage of the control) determined by acridine orange staining following treatment with cycloheximide in the presence of 0, 1, 10, 100 or 200 ng/ml of TIMP-1 as well as the results for cells treated with serum alone. * indicates p<0.001 (n=5). Panel C shows the level of caspase 3 activity (again expressed as a percentage of the control) in extracts from hepatic stellate cells treated with cycloheximide either in the presence of 0, 1, 10, or 100 ng/ml of TIMP-1 or of benzyloxycarbonyl-Val-Ala-Asp-fluoromethylketone. Results for a serum only control are also shown. * indicates p<0.001 (n=3). Panel D shows the results of TUNEL analysis to assess DNA fragmentation in hepatic stellate cells treated with cycloheximide in the presence or absence of TIMP-1. Results for a serum alone control are also shown. Results indicate the percentage of TUNEL positive cells with reference to the control. * indicates p<0.001 (n=2). Panel D shows a western blot for Bcl-2 protein on cell extracts from activated hepatic stellate cells induced to undergo apoptosis by cycloheximide treatment in the presence or absence of TIMP-1. Results for a serum only control are also shown.

FIG. 5 shows the proportion of apoptotic hepatic stellate cells (expressed as a percentage of the control) following treatment with Nerve Growth Factor (NGF) in the presence or absence of TIMP-1 in serum free conditions. * indicates p<0.02 for NGF treated alone versus NGF with TIMP-1 treatment (n=3).

FIG. 6 shows the proportion of apoptotic hepatic stellate cells (expressed as a percentage of the control) following incubation in serum-free conditions with 5% BSA for 18 hours in the presence or absence of neutralizing antibodies to TIMP-1 and also for cells treated with a nonimmune IgG control antibody. A serum alone control was also run. * indicates that p<0.0001 for hepatic stellate cells treated with neutralizing antibodies for TIMP-1 relative to nonimmune IgG control (n=3).

FIG. 7. Panel A shows the results of acridine orange staining and counting of apoptotic hepatic stellate cells following exposure to cycloheximide with or without wild type or T2G mutant TIMP-1. The results for a serum control are also shown. * indicates that p<0.01, NS indicates not significant (n=3). Panel B shows the caspase 3 activity of hepatic stellate cell extracts from cells treated with cycloheximide in the presence of wild type (active) TIMP-1 and the T2G mutant (which has no MMP inhibitory activity). * indicates that p<0.01 (n=3). Panel C shows the proportion of apoptotic hepatic stellate cells following exposure to cycloheximide in the presence or absence of either TIMP-1 (142.5 ng/ml; 5 nM) or the synthetic MMP inhibitor MMPI-1. * indicates p<0.001; ** indicates p<0.0001 (n=3).

FIG. 8. Panel A shows level of TIMP-1 mRNA expression as determined by Taqman quantitative PCR in total liver RNA. Level of expression was determined in rats treated with CCl₄ for 6 or 12 weeks and then either immediately assayed or allowed to recover for 15 days. PF0=peak fibrosis, immediately after the final injection of carbon tetrachloride; PF15 is after 15 days of spontaneous recovery. n=3 for each experiment group at each time point. All values have been normalized for GAPDH expression determined in parallel. Panel B shows the numbers of smooth muscle actin (SMA)-positive hepatic stellate cells in the livers of the rats. n=4 for each experimental group at each time point; ** indicates p<0.0001; * indicates p<0.03. Panel C shows a western blotting of whole liver homogenate of the rats for smooth muscle actin. Samples from untreated rats are also shown.

FIG. 9 shows histological analysis (Sirius Red stain) of rat livers harvested after 6 and 12 weeks of carbon tetrachloride intoxication twice weekly. Sections shown are from livers harvested at peak fibrosis (PF0) following 12 (Panel A) and 6 (Panel C) weeks of treatment and after a further 15 days of spontaneous recovery (Panels B and D respectively).

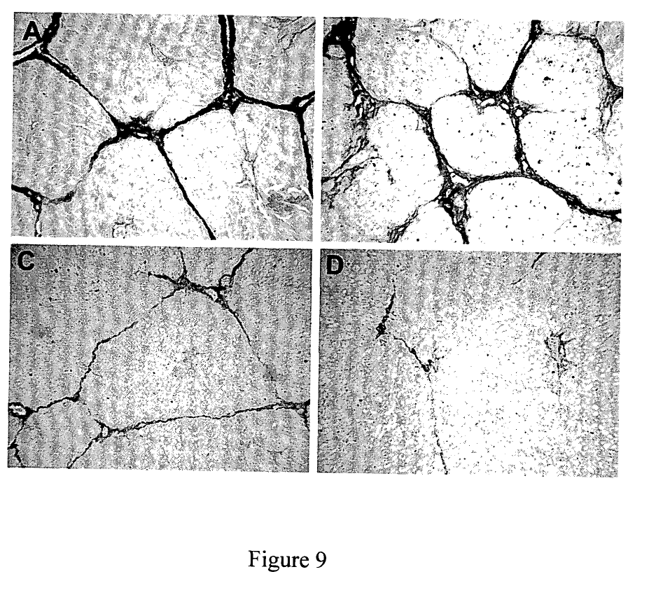

FIG. 10 shows the effect of a single injection of gliotoxin on liver sirius red staining after treatment for seven weeks with carbon tetrachloride. Rats were treated for seven weeks with carbon tetrachloride. One day after the final injection of carbon tetrachloride, rats were administered gliotoxin and killed after a further day. Panel A—control: liver section from a rat treated with vehicle (olive oil) for seven weeks and DMSO; Panel B—gliotoxin only: liver section from a rat treated with the vehicle (olive oil) for seven weeks and 3 mg gliotoxin/kg body weight; Panel C—carbon tetrachloride only: liver section from a rat treated with carbon tetrachloride for seven weeks and DMSO vehicle; and Panel D—carbon tetrachloride and gliotoxin: liver section from a rat treated with carbon tetrachloride for seven weeks and 3 mg gliotoxin/kg body weight. Results are typical of 5 separate animals.

### DETAILED DESCRIPTION OF THE INVENTION

The present invention is based on the finding that the selective induction of hepatic stellate cell apoptosis in vivo results in a reduction of the extent of fibrotic collagen in an animal model of liver fibrosis. This shows for the first time that inducing stellate cell apoptosis in vivo can be used as a way to treat liver fibrosis.

Hepatic stellate cells are thought to potentially play a role in the natural resolution of fibrosis. It is therefore surprising that the simultaneous elimination of a class of cells mediating a wound healing response in the liver does not result in a profound disturbance of hepatic structure and function, rather than the resolution of fibrosis which is seen.

In addition, although the liver has clearance mechanisms for the removal of apoptotic cells, these must have a finite limit in the number of apoptotic cells that they can successfully dispose of at any given time. If these clearance systems are overloaded it is possible that apoptotic cells which remained would undergo necrosis and cause damage to the surrounding tissues damaging liver function. The experiments provided here also show that the liver clearance mechanism of the liver for the removal of apoptotoic cells can cope with the additional numbers of apoptotic stellate cells generated when apoptosis of hepatic stellate cells is artificially stimulated.

The methods of the invention result in the selective apoptosis of hepatic stellate cells. It is preferred that apoptosis of other cell types in the liver, and indeed in the body, is not induced by the methods of the invention or that the level of apoptosis of other cell types is minimal in comparison to that of hepatic stellate cells. For example, it is preferred that the methods of the invention do not induce apoptosis of hepatocytes or other liver cell types cells as this might disturb liver function.

Subjects to be Treated

The subject to be treated will typically have, be developing, or be at risk of developing liver disease. In particular, the subject will be one that has, or is at risk of developing, liver fibrosis. The fibrosis may be at an early stage or may have progressed to a more advanced stage. In some cases the fibrosis may have progressed to such a stage that the individual has liver cirrhosis. The subject may also display inflammation in regions of their liver and there may be necrotic or degenerating cells present in the liver.

The liver of the subject will typically have a buildup of fibrotic extracellular matrix proteins. For example, these may include collagens and in particular type I, II and/or III collagens. Examples of other proteins which may be present in the fibrotic buildup include laminin, fibronectin and proteoglycans.

The liver disease, and in particular the liver fibrosis, in the subject may have a number of possible causes. The fibrosis may be due to infection with a pathogenic organism. For example, the fibrosis may be due to viral infection. In particular, the subject may be infected, or have been infected, with a virus which causes hepatitis. The subject may have chronic viral hepatitis. The virus may, for example, be hepatitis B, C or D virus. In some cases, and in particular where the subject has viral hepatitis, the subject may also be infected with HIV. It is possible, that the subject may have been, or be, infected with other organisms which cause liver fibrosis and in particular those which are present in the liver during some stage of their life cycle. For example, the subject may have, or have had, liver fluke.

The subject may have an inherited disease which causes, or increases the risk of, liver disease and in particular of liver fibrosis. For example, the subject may have one or more of hepatic hemochromatosis, Wilson's disease or α-1-antitrypsin deficiency. The subject may have an inherited disorder which causes some kind of structural or functional abnormality in the liver which increases the likelihood of liver fibrosis. The subject may be genetically predisposed to develop an autoimmune disorder which damages the liver and hence which can contribute to liver fibrosis.

In some embodiments of the invention, the subject to be treated may have liver disease due to a xenobiotic cause. For example, the subject may have been exposed to a chemical, drug or some other agent which causes liver damage and hence fibrosis. The subject may have been exposed to Rezulin™, Serzone™ or other drugs thought to cause liver damage and hence potentially liver fibrosis. The subject may be one who has had an overdose of a particular drug or exceeded the recommended dosage of a drug capable of causing liver damage. For example, the subject may have taken an overdose of paracetamol. The subject may have been exposed to chemicals which can cause liver damage such as, for example, at their place of work. For example, the subject may have been exposed to such chemicals in an industrial or agricultural context. The subject may have consumed plants which contain compounds which can cause liver damage, in particular this may be the case where the subject is an animal. For example, the subject may have consumed a plant containing pyrrolizidine alkaloid such as ragwort. The subject may have been exposed to environmental toxins thought to cause liver fibrosis.

The fibrosis may be alcohol induced. The subject may be, or have been, an alcoholic. The subject may have, or have been, consuming on average more than 50 units of alcohol per week, preferably more than 60 units of alcohol per week, more preferably more than 75 units of alcohol per week and even more preferably more than 100 units of alcohol per week. The subject may have been consuming such levels of alcohol for typically more than 5 years, preferably more than 10 years, more preferably more than 15 years and still more preferably for more than 20 years. In cases of alcohol induced fibrosis the subject may be aged, for example, over 25 years, preferably over 35 years, more preferably over 45 years and even more preferably over 60 years.

In other embodiments of the invention, the subject may have one or more of a number of other conditions known to result in liver fibrosis such as, for example, primary biliary cirrhosis, autoimmune chronic active hepatitis, and/or schistosomiasis. The subject may have or have had a bile duct blockage. In some cases, the underlying cause of the fibrosis may not be known. For example the subject may have been diagnosed as having cryptogenic cirrhosis.

Methods for diagnosing liver fibrosis and cirrhosis are well known in the art and in particular to clinicians and veterinarians in the field. Preferably, the subject will have been diagnosed as having a liver disease by a medical or veterinarian professional. The subject may display symptoms associated with liver disease such as one or more of jaundice, skin changes, fluid retention, nail changes, easy bruising, nose bleeds, and in male subjects may have enlargement of breasts. The subject may display exhaustion, fatigue, loss of appetite, nausea, weakness and/or weight loss.

The liver disease may have been, or be, confirmed by physical examination including techniques such as ultrasound. Liver biopsies may have been taken to look for buildup of fibrosis, necrotic cells, cellular degeneration and/or inflammation and other characteristic features of liver disease and in particular of liver fibrosis. Liver function may have been assessed in the subject to determine whether this is compromised in the subject. The nature and underlying cause of the liver fibrosis may be characterized. Any history of exposure to causative agents of liver fibrosis may be determined.

The subject to be treated may be any member of the subphylum chordata including, without limitation, a human or a non-human animal. The subject may be a non-human primate. The subject may be a chimpanzee or may be of another ape or monkey species. In a preferred embodiment of the invention, the subject to be treated is a human. The subject may be a farm animal including, for example, a cow or bull, sheep, pig, ox, goat or horse or may be a domestic animal such as a dog or cat. The subject may be a laboratory animal and in particular may be a rodent including, for example, a mouse, guinea pig, rat or hamster. The subject may be a bird. The subject may be any age, but will often be a mature adult subject.

Inducers of Apoptosis

The present invention provides methods for specifically inducing hepatic stellate cell apoptosis. The experimental evidence provided here demonstrates that this promotes or enhances the resolution of liver fibrosis. It is desired to induce apoptosis of hepatic stellate cells, but not of other cell types. Typically, this can be achieved by:
(i) by administering a selective inducer of apoptosis i.e. one which is capable of inducing hepatic stellate cell apoptosis, but not apoptosis of the other cell types that the inducer will come into contact with; or
(ii) by delivering an inducer of apoptosis specifically to hepatic stellate cells, but not to other cell types of the subject.

Alternatively, selective induction of hepatic stellate cell apoptosis may be achieved by administering an agent that can give rise to an inducer of hepatic stellate cell apoptosis in the subject to be treated, where:
(i) the agent is specifically delivered to hepatic stellate cells;
(ii) the agent gives rise to a selective inducer of hepatic stellate cell apoptosis; and/or
(iii) the agent only gives rise to the inducer of apoptosis in hepatic stellate cells.

In many embodiments of the invention the agent will comprise a nucleic acid molecule which can be transcribed to give rise to a polypeptide or RNA inducer of hepatic stellate cell apoptosis.

In some embodiments of the invention any of the above methods, and in particular ways to ensure selectivity, may be combined to ensure higher levels and preferably maximal selectivity. Thus the inducer may be specifically delivered to stellate cells and also be only capable of inducing stellate cell apoptosis. Similarly the agent may only be delivered to hepatic stellate cells and/or only be capable of giving rise to the inducer in hepatic stellate cells.

A further factor which can be used to facilitate selectivity may be that the inducer or agent is administered in such a way that it only reaches a localized region of the body at a significant concentration. Preferably, the inducer or agent may be administered specifically to the liver. For example, the inducer or agent may be delivered via the hepatic portal vein. Alternatively, the inducer or agent may be delivered intraperitoneally and hence, although a larger proportion of the body will be exposed to the inducer or agent, the whole body will not be.

In some embodiments of the invention the inducer or agent may be administered via an implant. The implant may be inserted into the liver or surrounding area to ensure that the inducer or agent is released locally to the liver. In particular, the implant may be placed in an area of the liver which is fibrotic or adjacent to such an area. The implant may be placed in, or adjacent to, an area where fibrotic buildup is at its highest. Typically, the implant will be inserted by surgical means. Multiple implants may be introduced into several areas of the liver. Typically, the condition of the subject, and in particular the severity of the liver disease, will be assessed to help decide when to insert the implant into the subject. In some cases, a further implant may be inserted after the active life of a previous implant has finished. In such cases, the further implant may, for example, be inserted immediately, or shortly after, the active life of the previous implant has finished or it may be inserted when the liver fibrosis begins to increase, or shows no further regression, in the subject to be treated.

The implant may be in any suitable form. It may, for example, take the form of a three dimensional martix, membrane or other such structure. The implant may be in the form of a solid structure. It may be porous to help faciliate release of the inducer or agent. The inducer or agent itself, or a composition comprising it, may be coated onto an implant. The implant itself may comprise the inducer or agent. The implant will comprise any suitable biocompatible material. The implant, parts of the implant, or coatings on the implant may be designed to breakdown gradually to slowly release the inducer or agent into the surrounding tissues. The implant may comprise or be coated with an alginate. Suitable implants and techniques for the generation of implants are known in the art and may be employed in the invention. The implant may be used to deliver any inducer or agent of the invention or indeed any other molecule of the invention.

The implant will typically be designed to release the inducer or agent at a chosen rate. For example, in some cases, it may be desired to release the inducer or agent from the implant into the surrounding tissues over a prolonged period such as for more than a month, preferably for more than two months and even more preferably for more than six months. Prolonged release of the inducer or agent from the implant may, for example, be desired where the subject has been suffering from chronic liver disease, typically over a prolonged period, and in particular where the subject is likely to continue to be exposed to the stimulus responsible for the liver fibrosis. In other cases the implant may be designed to release the inducer or agent over a shorter period, such as, for example, for less than a month, preferably less than two weeks and even more preferably for less than a week. In some cases a further implant may be inserted after the first implant has ceased to release an effective amount of the inducer or agent. This replacement may be periodically. Alternatively, the further implant may be inserted at a time when fibrosis begins to progress again or at least is no longer regressing.

The implant will typically be designed to release a chosen concentration of inducer or agent into the surrounding tissues. The implant will preferably be designed to deliver an effective amount of inducer or agent to the liver and in particular to the fibrotic tissue. Preferably, the implant will be designed so that the concentration of the inducer or agent released is such that apoptosis will only be induced within a given radius. For example, the concentration of inducer or agent released may be such that apoptosis of cells is only be induced in the liver or in a fibrotic portion of the liver. This will allow the exposure of regions outside the liver, or healthy regions of the liver, to the inducer or agent, and in particular the inducer, to be minimized.

By administering the inducer or agent only to the liver, or to a localized region including the liver, this will mean that the subset of cell types that the inducer or agent is exposed to is reduced. This means that the inducer administered or generated only has to be incapable of inducing apoptosis of a smaller subset of cell types to ensure that it does not have undesired side effects. For example, it may mean that the inducer administered or generated only has to discriminate between hepatic stellate cells and other cell types present in the liver or that the means of delivery only has to discriminate between these cell types.

In a preferred embodiment, the method of the invention will induce apoptosis of hepatic stellate cells, but not of any other cell type in the body of the subject. Preferably, the inducer administered or generated will induce apoptosis of activated hepatic stellate cells i.e α-smooth muscle actin positive hepatic stellate cells.

Typically, the inducer will be capable of inducing apoptosis in hepatic stellate cells, but not other cell types of the liver and/or will be delivered to, or generated in, hepatic stellate cells, but not to other liver cell types. Preferably, the inducer will not induce apoptosis of other cell types present in the liver such as, for example, infiltrating immune cells. Preferably, therefore, the inducer will not be capable of inducing apoptosis of, or not be delivered to, or generated in, one or more of, or more preferably all of, hepatocytes, Kupffer cells, epithelial cells, sinusoidal endothelial cells, pit cells, biliary endothelial cells, Mast cells and T lymphocytes.

Preferably, the inducer will not stimulate apoptosis of immune cells present in the liver such as macrophages, lymphocytes and/or neutrophils. In an especially preferred embodiment the inducer will be a selective inducer capable of inducing hepatic stellate cell apoptosis, but not apoptosis of any other cell type in the body of the subject and/or will be delivered in such a way that it is only targeted to hepatic stellate cells and not other cell types in the body of the subject. The inducer may only be generated in hepatic stellate cells because of the agent employed.

In some embodiments, the inducer may cause apoptosis of one or more other cell types, in addition to apoptosis of hepatic stellate cells, but apoptosis of the other cell types will be at a lower level than that of hepatic stellate cells. For example, the level of hepatic stellate cell apoptosis may be at least two fold, preferably at least five fold, more preferably at least ten fold, even more preferably at least 50 fold and still more preferably at least 100 fold greater than that of another cell type when the stellate cell and second cell type are exposed to equivalent concentrations of the inducer. The level of selectivity may be more than 500 fold, preferably more than 1000 fold, even more preferably more than 10,000 fold and most preferably the inducer will be absolutely selective for hepatic stellate cells. Such levels of selectivity may refer to values determined in vitro and/or in vivo. They may refer to specificity with regard to hepatic stellate cells and any other particular cell type or all cell types of the body of the subject. They may refer to hepatic stellate cells and any other liver cell type or all liver cell types. Preferably, such levels of selectivity will be displayed with regards to hepatocytes.

It may also be that a particular inducer administered or generated will be selective, or most selective, at a particular concentration. Thus titrations can be performed to determine what proportion of hepatic stellate cells are induced to undergo apoptosis at a particular concentration of inducer. This can also be determined for other cell types and the values for hepatic stellate cells and other cell types compared. The concentration at which the inducer is most selective for hepatic stellate cells and gives a high level of hepatic stellate cell apoptosis may then be picked. The concentration of inducer or agent administered may be chosen accordingly. Thus a concentration at which the level of apoptosis in the second cell type is 50% or less, preferably 25% or less, more preferably 10% or less, even more preferably 5% or less, still more preferably 1% or less and yet more 0.1% or less than the level of apoptosis of hepatic stellate cells may be employed. These tests may be carried out in vitro and/or in vivo.

The inducer of apoptosis may act in a number of ways. Hepatic stellate cells may naturally be exposed to stimuli or molecules whose effect is to reduce the possibility of them undergoing apoptosis. In effect, the stellate cell receives a signal to tell it not to undergo apoptosis or which decreases the chance of the cell undergoing apoptosis. The inducers of the present invention may block such a signal and hence promote apoptosis of hepatic stellate cells.

The experimental results presented herein show that tissue inhibitors of matrix metalloproteases (TIMPs) may decrease the probability of hepatic stellate cells undergoing apoptosis by their effect on matrix metalloproteases (MMPs). Thus, by preventing the interaction of TIMPs with MMPs this will result in an increase in apoptosis of stellate cells. This may be achieved in a number of ways. For example, the level of TIMP expression may be downregulated, hence there will be less inhibition of MMP activity and conversely more hepatic stellate cell apoptosis. Alternatively, a molecule capable of preventing or reducing the interaction of a TIMP with an MMP may be employed. Another possibility is that the actual level of MMP expression may be increased. In a preferred embodiment of the invention the TIMP which will be targeted is TIMP-1.

The level of TIMP expression may be down-regulated using techniques such as antisense RNA, siRNA (short inhibitory RNA) and/or a catalytic RNA specific for the TIMP transcript. These may be expressed from constructs introduced into the liver and preferably targeted specifically to hepatic stellate cells. They may be expressed from hepatic stellate specific promoters to ensure that TIMP expression is specifically down regulated in these cell types alone. Alternatively, other molecules capable of downregulating TIMP expression may be administered. These may be naturally occurring molecules capable of downregulating TIMP expression or may be synthetically generated molecules capable of downregulating TIMP expression. For example, libraries may be screened to identify molecules capable of down regulating TIMP expression and these may then employed in the invention.

In respect of antagonists of the interaction of TIMPs with MMPs these may be substances such as antibodies or derivatives thereof, or may be other substances such as small chemical molecules. The assays provided herein may be used to screen large numbers of substances to determine if they are capable of modulating the interaction between MMPs and TIMPs and in particular if they can promote apoptosis of hepatic stellate cells. Such modulators may then be introduced into the liver, or alternatively nucleic acid constructs capable of expressing or generating them may be introduced, in order to inhibit the interaction of TIMPs with MMPs. Preferably, the molecules themselves may be introduced as this will help to give control over the length of time the interaction between TIMPs and MMPs is inhibited for and prevent excessive MMP activity.

In another embodiment of the invention, the inducer will be one whose action is to induce apoptosis itself rather than to antagonize a molecule which is preventing apoptosis. Thus the inducer may trigger a pathway which leads to the apoptosis of hepatic stellate cells. For example, the inducer may bind to a receptor on a hepatic stellate cell which results in the bound cell undergoing apoptosis.

In one embodiment of the invention the inducer may bind to the p75 receptor which is present on hepatic stellate cells. The binding of p75 will trigger apoptosis of the stellate cells. P75 is a receptor for Nerve Growth Factor (NGF) and the molecule which will be used to bind p75 may be nerve growth factor or a derivative thereof capable of binding to the receptor and stimulating apoptosis. The molecule used to bind to the receptor may be an antagonist of the receptor which is capable of binding to the receptor and triggering apoptosis. The antagonist may be an antibody or derivative thereof or another substance capable of binding the receptor to induce apoptosis. Again, the assays of the invention may be modified to screen large numbers of candidate substances. As p75 is only expressed in the liver on hepatic stellate cells, but is expressed elsewhere in the body, preferably either the antagonist of p75 will be administered locally to the liver and/or will be specifically delivered to stellate cells using the methods of the invention.

In some embodiments of the invention, the inducer will not act on a receptor, but will act on a molecule downstream of the receptor. Thus, the end result may be the same as antagonizing the receptor, but a downstream target, such as a molecule in the signal transduction pathway of the receptor, is selected as a target.

In one embodiment of the invention the inducer employed will antagonize a 5HT₂ receptor present on the surface of hepatic stellate cells and hence induce the cells to undergo apoptosis. Alternatively, the inducer may act downstream of the 5HT₂ receptor to induce an equivalent effect to antagonizing the 5HT₂ receptor directly. By acting downstream of the receptor this may mean that cell specific delivery or expression of the antagonist can ensure that only hepatic stellate cells are induced to undergo apoptosis.

In a preferred embodiment of the invention the antagonist will act on, or downstream of, the 5HT_{2B} receptor, as this receptor is expressed on activated hepatic stellate cells but not hepatocytes. In such embodiments, the inducer or agent may be delivered locally to the liver to minimize exposure of other cell types in the body expressing the receptor to the inducer. Preferably, the inducer will not bind and/or antagonize other 5HT₂ receptor subtypes, only binding and antagonizing the 5HT_{2B} receptor subtype or will have a high degree of selectivity for the 5HT_{2B} receptor subtype. For example, the antagonist may bind and/or activate the 5HT_{2B} receptor subtype twice, four fold, ten fold, 100 fold, 1000 fold or more readily than other 5 HT₂ receptor subtypes and in particular than the 5HT_{2A} receptor subtype. The antagonist, and/or method employing it, may have any of the degrees of selectivity mentioned herein.

In some embodiments the inducer may act on, or downstream of other 5HT₂ receptor subtypes in addition to, or alternatively to, the 5HT_{2B} receptor subtype. In one embodiment the inducer may act on the 5HT_{2A} receptor subtype, or downstream of it, but be delivered in such a way, or selectively expressed, to ensure that only hepatic stellate cells are induced to undergo apoptosis.

The 5HT₂ antagonist employed may be the natural ligand for the receptor, such as serotonin. In some cases the antagonist may be a derivatized version of serotonin specifically capable of binding the 5HT_{2B} receptor subtype. In some cases the antagonist may be an artificial antagonist of a 5HT₂ receptor and in particular of a 5HT_{2B} receptor subtype. Such artificial atangonists may be identified using methods well known in the art such as by screening libraries as discussed further below.

In other embodiments of the invention the inducer may trigger mitochondrial permeability transition (MPT) and/or calcium flux. The inducer may inhibit the activity of the factor NF-kB or other factors thought to play a role in control whether or not stellate cells undergo apoptosis or not. The inducer may act on IkB, which is an inhibitor of NF-kB function. In particular, it may increase the levels of IkB present in the hepatic stellate cell and hence downregulate NF-kB function. In some preferred embodiments the inducer may inhibit the degradation of IkB. The inducer may inhibit the expression of, or activity of, Bcl-2 or alternatively it may promote the activity of a caspase and in particular of caspase 3.

In one embodiment of the invention the inducer employed may be sulfasalazine [2-hydroxy-5-[-4-[C2-pyridinylamino)sulfonyl]azo]benzoic acid] or a derivative thereof capable of inducing hepatic stellate cell apoptosis. In some embodiments the inducer may be a derivative of sulfasalazine such as 5 aminosalicyclic acid (5-ASA), 4 aminosalicyclic acid (4-ASA). In other embodiments the derivative may be sulfapyridine. Derivatives of 5 aminosalicyclic acid (5-ASA), 4 aminosalicyclic acid (4-ASA) and/or sulfapyridine capable of inducing hepatic stellate cell apoptosis may also be employed in the invention. Again, selectivity may be ensured by selectively delivering the sulfasalazine, or derivative thereof, to the hepatic stellate cell. In many embodiments, sulfasalazine or the derivative will be administered to the liver rather than to the whole of the body.

In some embodiments of the invention the specific induction of hepatic stellate cell apoptosis will be achieved using a selective inducer of hepatic stellate cell apoptosis or delivering an agent capable of giving rise to such an inducer. A selective inducer of hepatic stellate cell apoptosis is an inducer which induces apoptosis of hepatic stellate cells, but which does not induce apoptosis of a second cell type. Preferably, the inducer will not induce apoptosis in any other cell type apart from hepatic stellate cells or at least will not induce apoptosis in the other cell types which will be exposed to the inducer in the methods of the invention. The level of specificity for hepatic stellate cells may be, for example, any of those specified above.

The inducer may be selective for stellate cell apoptosis because it binds to a molecule only found on hepatic stellate cells. This binding may actually induce apoptosis itself or ensure internalization into the cell where the inducer can then cause apoptosis to occur. Alternatively, the inducer may be one which is capable of gaining entry to all cell types, but only causes apoptosis in hepatic stellate cells. This may be because its target is only present in hepatic stellate cells.

Selective inducers of hepatic stellate cell apoptosis may be identified by employing the assays of the invention. In some cases a first screen may be used to identify substances which are capable of inducing hepatic cell apoptosis and this may be followed by a second screen of those substances capable of inducing hepatic stellate cell apoptosis to identify those which do not induce apoptosis of other cell types. Although, entire libraries of candidate substances may be screened in some cases rational design of inducers may be employed to help develop selective inducers and to streamline the process. Such rational design may employ a known inducer of hepatic stellate cell apoptosis as a starting point.

In one embodiment of the invention the selective inducer of apoptosis may be gliotoxin or a derivative thereof which is capable of inducing hepatic stellate cell apoptosis, but not, preferably, apoptosis of other cell types and, in particular, not apoptosis of other liver cell types. Preferably, the derivatives of gliotoxin employed will retain the disulphide bridge of gliotoxin. The ability of gliotoxin derivatives to selectively induce stellate cell apoptosis may be assessed using the methods discussed herein and in particular the ability to induce apoptosis of hepatic stellate cells and hepatocytes may be determined and compared.

In many embodiments of the invention the inducer of apoptosis, or the agent capable of giving rise to it, will be specifically delivered to hepatic stellate cells to ensure that it is only these cells which are triggered to undergo apoptosis. The selective delivery of the inducer or agent to stellate cells may be achieved in a number of ways.

The inducer or agent may be packaged or encapsulated in a variety of ways. For example, the inducer or agent may be present in, or comprise, a liposome or viral particle. The agent capable of giving rise to the inducer may be a nucleic acid molecule which can be transcribed to give rise to the inducer or a polypeptide molecule capable of generating an inducer. For example, the agent may comprise a nucleic acid which is packaged into a viral particle or liposome. The virus or lipsome may specifically deliver the agent to hepatic stellate cells and not any of the other cell types that it comes into contact with and hence the inducer will only be generated in these cell types.

The particle which the inducer or agent is present in, is conjugated to, or comprises may have a ligand present which binds a molecule found on the surface of hepatic stellate cells and which is preferably only found on hepatic stellate cells. This may ensure that the particle specifically binds and allows entry of the inducer or agent into hepatic stellate cells.

In some embodiments of the invention the inducer may be encoded by, or transcribed from, a nucleic acid and the nucleic acid administered to the subject, rather than the inducer itself. The inducer in such embodiments may be a polypeptide or RNA molecule. The nucleic acid may be specifically delivered to hepatic stellate cells or alternatively may be delivered to a wider range of cell types, but only be expressed in hepatic stellate cells. This may be due to the presence of a hepatic stellate cell specific promoter or other hepatic stellate cell specific regulatory element being operably linked to the nucleic acid molecule encoding the inducer or from which the inducer is transcribed.

In some cases the inducer expressed or transcribed from the nucleic acid may be a selective inducer of hepatic stellate cell apoptosis and hence the nucleic acid can be delivered to a wider range of cells, but only give rise to apoptosis in hepatic stellate cells.

In some embodiments of the invention the inducer may be an antisense RNA molecule capable of inducing apoptosis of hepatic stellate cells. Such antisense RNA molecules may be administered directly to the subject or alternatively an agent comprising a nucleic acid molecule which can be transcribed to give such an antisense molecules may be administered to the subject operably linked to an appropriate promoter. In some embodiments of the invention the inducer may be a siRNA (short interfering RNA) molecule capable of inhibiting the expression of a gene in hepatic stellate cells which results in the induction of apoptosis. Preferably, such siRNAs will selectively trigger hepatic stellate cell apoptosis. In other embodiments the inducer may be a catalytic RNA capable of preventing or inhibiting expression of a gene in hepatic stellate cells, where the inhibition results in apoptosis of the hepatic stellate cell. Again the catalytic RNA may be delivered directly or transcribed from a nucleic acid administered to the subject. The catalytic RNA may be a Ribozyme.

In embodiments of the invention which involve the administration of a nucleic acid which has to be transcribed in order to give rise to an inducer often a cell specific promoter, regulatory element and/or enhancer will be employed to ensure that the nucleic acid is only expressed in hepatic stellate cells. The promoter, regulatory elements and/or enhancer may be totally hepatic stellate cell specific or, out of the cell-types that the nucleic acid is to be delivered to in the subject, will only be expressed in hepatic stellate cells.

In some cases, the hepatic stellate cell specific promoter, regulatory elements or enhancer may give rise to expression in other cell types but at a much lower level and/or frequency than in hepatic stellate cells. For example, the level of expression in other cell types, including any one or more of those mentioned herein, may be less than 10%, preferably less than 5%, even more preferably less than 1%, still more preferably less than 0.1% and yet more preferably less than 0.01% of that seen in hepatic stellate cells. The level of expression, may be, for example, may be determined by techniques such as blotting and/or quantitative RT-PCR. Alternatively, the level of protein expression may be used to determine the specificity of expression in hepatic stellate cells.

Techniques for identifying cell-specific promoters, such as differential display and subtractive hybridization, are well known in the art and may be employed to identify promoters, regulatory elements or enhancers with any of the levels of specificity mentioned herein, and in particular which are hepatic stellate cell specific, for use in the invention. The ability of a promoter or regulatory element to give rise to a particular specificity of expression, and in particular to give rise to hepatic stellate cell specific expression, may be confirmed by transfecting a construct comprising the promoter/regulatory element operably linked to a reporter gene into a range of cells in vitro and then detecting in which cell types the reporter gene is expressed. Typically, the range of cells transfected will include hepatic stellate cells and other liver cell types including any of those mentioned herein.

Alternatively, the specificity of a promoter and/or regulatory element may be assessed in vivo, again by using a reporter gene. The construct may be introduced as a transgene or alternatively introduced into an adult animal. Any suitable technique for delivering nucleic acids to cells in vivo may be employed in order to assess the specificity of expression achievable using the construct.

In some embodiments the promoter operably linked to the region encoding the inducer will be inducible. This may be in addition to being a cell specific promoter or as an alternative to it. This may add a further level of control over apoptosis of hepatic stellate cells. The compound or stimulus necessary to induce the promoter may then be administered locally and/or at a specific chosen time.

Nucleic acids encoding an inducer of hepatic stellate cell apoptosis may be delivered by any suitable method. Methods for delivering nucleic acids to specific target cells are well known in the art and may be employed in the invention. The nucleic acid may, for example, be delivered in the form of a liposome or a viral particle. The nucleic acid may be administered as a naked nucleic acid molecule. In some embodiments nucleic acids may be administered as nucleic acids coated onto suitable particles. Methods for delivering particles coated with nucleic acids are well known in the art and may be employed. For example, various needleless syringes which use high velocity jets of gas to deliver particles coated with nucleic acid are known and may be employed to deliver constructs of the invention.

The inducer may be delivered to the liver using a virus which displays tropism for the liver and in particular for hepatic stellate cells. In such emodiments, typically the virus will comprise a polynucleotide encoding the inducer. Any of the nucleic acid molecules discussed herein may be delivered using a virus. The infection of the target cell with the virus will lead to the expression of the inducer in the target cell and hence apoptosis of the target cell. Any suitable virus may be employed. Such viruses may be prepared by methods well known in the art. In one embodiment a recombinant adenovirus may be employed which is capable of infecting hepatic stellate cells. In some embodiments the virus employed may infect a wider range of cells than just stellate cells, but the gene encoding the inducer may only be expressed in hepatic stellate cells due to the promoter and/or regulatory elements chosen to drive expression of the inducer. The virus chosen to deliver the inducer may give rise to any of the levels of specificity specified herein and any of the nucleic acid molecules discussed herein may be delivered via a virus.

The nucleic acid may be delivered via any suitable route. In some embodiments the nucleic acid molecule may be delivered to the target area during surgery. For example, the nucleic acid may be delivered to the liver and/or its surrounding tissues during surgery and typically when the target area is exposed or more readily accessible. The nucleic acid may be delivered via a blood vessel and in particular via the hepatic portal vein. The delivery mechanism for the nucleic acid molecule may ensure that the nucleic acid is specifically delivered to hepatic stellate cells. For example, in the case of lipsomes, viruses and other embodiments where the nucleic acid is packaged and/or encapsulated in some form, molecules may be present on the surface of the delivered particle to target it to hepatic stellate cells. Typically, such targeting molecules will only bind a molecule specifically present on hepatic stellate cells such as a receptor.

In embodiments of the invention which employ nucleic acids, suitable nucleic acid derivatives may also be employed. For example various DNA and RNA analogues molecules which are less readily degraded or which have other preferable properties are well known in the art and may be employed.

In some embodiments of the invention due to the ability of the method employed to specifically induce hepatic stellate cell apoptosis, the inducer or agent will not have to be administered locally to the liver, but can be administered via a route which results in a wider range of cells being exposed to the inducer or agent. For example, the agent or inducer may be administered via the intravenous route.

In some embodiments of the invention the inducer may be in the form of a prodrug, i.e in an inactive form, which can then be processed to give rise to an inducer. The prodrug may completely lack the ability to induce hepatic stellate cell apoptosis or may have much reduced activity, such as less than 10%, preferably less than 1%, more preferably less than 0.1% and even more preferably less than 0.01% of the activity of the actual inducer. Typically, the inactive form may be converted into the inducer enzymatically. For example, the inactive form may be a polypeptide which can be proteolytically cleaved at a specific site to give rise to the inducer. The inactive form may be a chemical or other substance which has to be cleaved or modified in some way to render it active.

Activation may involve reaction of the inactive form of the inducer with a second, or further, molecules. Alternatively, an active inducer may be formed from two, or more, molecules reacting with each other. Formation of the active form of the inducer may involve modification of a molecule by addition or removal of groups such as, for example, phosphate groups or methylation.

The invention also includes embodiments where an agent capable of generating or giving rise to an inducer of hepatic stellate cell apoptosis is administered rather than the inducer itself. Thus in the absence of the agent the inducer of hepatic stellate apoptosis is not generated or is generated at a much reduced level such as at less than 50%, preferably less than 25%, more preferably less than 5%, even more preferably at less than 1% and still more preferably at less than 0.1% of the level generated when the agent is present.

The agent may be an enzyme which converts the inactive form of the inducer into an active form. The agent may be a nucleic acid encoding such an enzyme. Alternatively, the agent may produce the inducer enzymatically from one or more substrates. The substance which the agent acts on may also be administered or may be an endogenous molecule. Thus in some embodiments of the invention it may be the agent alone which is administered to the subject and the molecules it acts on to generate the inducer of apoptosis will already naturally occur in the subject. In other embodiments of the invention the agent may be a substance which a naturally occurring enzyme found in the subject can act on in order to generate an inducer.

It will be apparent that there a number of ways of ensuring that hepatic stellate cells are specifically induced to undergo apoptosis. The invention encompasses any combination which results in the specific induction of hepatic cell apoptosis. Thus it may be that the agent administered may be one or more of an activating agent, pro-inducer and/or compound from which an inducer is generated or is necessary for the generation of the inducer. One or more of the activating agent, pro-inducer, and/or compound from which an inducer is generated or necessary for generation of the inducer may be present endogenously. Any suitable combination may be employed in the invention as long as it results in the selective induction of hepatic stellate cell apoptosis. Any two or more ways mentioned herein for specifically inducing hepatic stellate cell apoptosis may be employed in combination to increase the level of selectivity.

In some embodiments of the invention the specific induction of hepatic stellate cells may be achieved, or be contributed to, because a substance necessary for the administered agent to give rise to an inducer of hepatic stellate cell apoptosis only occurs locally in the liver, and in particular only in hepatic stellate cells.

Tests to assess the ability of a specific method of the invention to selectively induce hepatic stellate cell apoptosis may be carried out using any suitable assay or model. Such assessment may be in vitro or in vivo. In some embodiments the tests may be carried out on normal animals and/or cells. For example, such tests may be carried out on a rodent and in particular on a rat. In other embodiments the efficacy of a particular method may be assessed in a model of liver fibrosis and in particular in an in vivo model, such as an animal model, preferably a rodent model and even more preferably a rat model. In particular, a model of chronic liver fibrosis may be employed.

The model of liver fibrosis employed will typically involve the administration of a compound capable of inducing liver fibrosis to the animal. Alternatively, surgical procedures may be performed on the animal which induce fibrosis. The model may involve the administration of carbon tetrachloride to the animal. For example, carbon tetrachloride may be administered once, twice or more per week for a period of from five to fifteen, preferably from six to twelve and even more preferable for from eight to ten weeks in order to induce liver fibrosis.

The inducer or agent of the invention may be administered at the same time as the agent inducing the fibrosis or during the period in which the agent inducing fibrosis is being administered to the animal. The two may be administered in the same or separate compositions. Alternatively, the inducer or agent may be administered after the administration of the inducer of fibrosis has ceased. Typically, controls will also be carried out where no inducer, agent capable of giving rise to an inducer of apoptosis and/or agent capable of causing liver fibrosis is administered. The control animals may be treated with the vehicle which was employed for the administration of the inducer and/or agent, but with no actual inducer or agent present. For example, the control animals may be administered olive oil alone.

In some embodiments of the invention the inducer administered to, or generated in, the subject may not have been previously known to be an inducer of hepatic stellate cell apoptosis. New inducers of hepatic stellate cell apoptosis can be identified by methods well known in the art and in particular by screening libraries. The method may first identify substances capable of inducing hepatic stellate cell apoptosis and then screen the identified substances to identify those which do not induce apoptosis in other cell types. Alternatively, inducers capable of inducing apoptosis in a wider range of cells types, including hepatic stellate cells, which can then be selectively delivered to hepatic stellate cells will be identified by screening libraries.

Any of the assay methods mentioned herein may employed and in particular any of the methods mentioned herein for identifying apoptotic cells may be employed in the assay. Typically, the initial screening steps will be carried out in vitro. Once an inducer is identified its efficacy in vivo can be determined. For example, its efficacy in healthy animals and/or in an animal model of liver fibrosis may be determined. In particular the carbon tetrachloride model of liver fibrosis discussed herein may be employed.

Suitable test substances which can be tested to identify inducers of hepatic stellate cell apoptosis include combinatorial libraries, defined chemical entities and compounds, peptide and peptide mimetics, oligonucleotides and natural product libraries, such as display (e.g. phage display libraries) and antibody products. Subtances may be based on the structure of a known inducer of hepatic stellate cell apoptosis and variants produced, for example, by mutagenesis and/or rational design.

In some cases the substances which will be screened will be variants of a substance capable of inducing hepatic stellate cell apoptosis, but which which also induces apoptosis in other cell types, in order to identify variants with greater selectivity for inducing apoptosis in hepatic stellate cells. In some case, variants of a substance which is already a selective inducer of hepatic stellate cell apoptosis may be tested to identify variants with greater specificity and/or other preferred properties such as reduced toxicity.

Typically, organic molecules will be screened, preferably small organic molecules which have a molecular weight of from 50 to 2500 daltons. Candidate products can be biomolecules including, saccharides, fatty acids, steroids, purines, pyrimidines, derivatives, structural analogs or combinations thereof. Candidate agents are obtained from a wide variety of sources including libraries of synthetic or natural compounds. Known pharmacological agents may be subjected to directed or random chemical modifications, such as acylation, alkylation, esterification, amidification, etc. to produce structural analogs.

Test substances may be used in an initial screen of, for example, 10 substances per reaction, and the substances of these batches which show ability to induce hepatic stellate cell apoptosis tested individually. Test substances may be used at a concentration of from 1 nM to 1000 μM, preferably from 1 μM to 100 μM, more preferably from 1 μM to 10 μM. Preferably, the activity of a test substance is compared to the activity shown by a known inducer.

In some cases the assay will assess binding by test substances to a specific target molecule, the binding of which is known to induce hepatic stellate cell apoptosis. Suitable assays for identifying such binding are well known in the art and may be employed. Substances which bind can then be assessed for their ability to activate hepatic stellate cell apoptosis. The ability of the substance to bind to cell types other than hepatic stellate cells and/or to induce hepatic stellate cell apoptosis in such cell types may be assessed. In other embodiments, the ability of a test substance to modulate the activity of a known target molecule which controls whether or not a hepatic stellate cell undergoes apoptosis may be assessed. Such assays may be cell based or may be cell free.

Any of the inducers identified by the methods discussed herein may then either be delivered directly to the subject or an agent capable of generating the agent may be administered.

Subject Assessment

Hepatic stellate cell apoptosis and the resolution of liver fibrosis may be assessed in the subject using a number of techniques. Overall improvement in the liver disease that the subject is suffering from may also be seen. The condition of the subject and liver function in the subject may be assessed. Thus the subject may be assessed to monitor any lessening in the severity of, or the disappearance altogether, of one or more symptom associated with liver disease and in particular with liver fibrosis. For example, whether or not there is any change in jaundice, fluid retention, ease of bruising, frequency of nose bleeds, skin or nail condition may be assessed. The general well being of the subject may improve and this may be assessed as an indicator of recovery. Thus the subject may display increased appetite, reduction in the incidence, or severity of, nausea, increase in weight and/or general feelings of strength and energy. The subject may also have reduced incidence of hospitilization or need of other medical attention.

The liver function of the subject may be improved or increased. Liver function may be stabilized. This may be assessed in a variety of ways. Liver biopsies or blood samples may be taken and markers of liver function may be determined. Markers of liver function which may be studied include hyaluronic acid, procollagen IIIN peptide, procollagen IC peptide, Undulin-collagen 16, 7S type IV collagen, MMP-2 and TIMP-1 levels.

The subject's liver may show decreased nodulization, necrosis and/or inflammation. In particular, the liver of the subject may display a decrease, or stabilization, in the amount of fibrosis in their liver. The presence of fibrotic material in the liver may be decreased and this may be determined by staining sections from liver biopsies using stains such as Sirius red. The presence and amount of particular fibrotic extracellular matrix components such as, for example, collagens and in particular collagens I and III may be determined. Biochemical analyses may also be carried out to determine levels of TIMPs and/or MMPs and the reduction of TIMP expression in the subject.

The apoptosis of hepatic stellate cells in the liver may also be determined from liver biopsies. Any change, and in particular any increase, in the frequency of apoptosis of hepatic stellate cells may be measured. Apoptotic cells can be identified using a number of well known methods. Techniques such as TUNEL staining (terminal deoxynucleotidyl transferase mediated deoxyuridine trisphosphate nick end labelling) may be used to identify apoptotic cells. TUNEL staining is particular useful as it may be used to identify apoptotic cells in situ. Through co-staining it can be checked that the cells undergoing apoptosis are hepatic stellate cells such as by staining for α-smooth muscle actin expressing cells.

Other well known techniques for identifying and/or quantifying apoptosis may be employed such as, for example, Annexin V staining, antibodies against single stranded DNA, caspase substrate assays, ligation mediated PCR and cell membrane permeability staining. DNA fragmentation may be analyzed by gel electrophoresis. Staining may also be used to determine the morphological characteristics associated with apoptosis, such as membrane blebbing and the breakdown of the nucleus. Acridine orange staining may be used to identify apoptotoic cells. Cells may be stained with propidium iodide to analyze DNA content. Tests such as trypan blue staining may be used to check that the membrane cell is intact and that they are apoptotic not necrotic.

For medicaments and methods of the invention which involve the administration of a nucleic acid, various techniques well known in the art may be used to assess expression from the administered nucleic acid. For example, northern blotting and/or RT-PCR may be used to study expression at the RNA level. Such analysis may be carried out on tissue recovered from the subject and typically on tissues from the liver of the subject. Techniques for taking liver biopsies are well known in the art and may be employed. In some cases in situ PCR may be performed on tissue sections to allow identification of both hepatic stellate cells and cells expressing the nucleic acid construct. Typically, the two should be one and the same. Alternatively, specific cell types may be separated from the recovered tissue to analyse which cell types are expressing the nucleic acid.

The presence of the inducer may be identified in tissues recovered from the subject and in particular on liver tissue recovered from the subject. Techniques such as western blotting may be employed to determine the presence and location of the protein. The tissue may also be stained with for hepatic stellate cell specific markers in order to demonstrate that the inducer is localised to hepatic stellate cells.

Pharmaceutical Compositions and Administration

The inducers and agents for use in the methods of the invention may be formulated with standard pharmaceutically acceptable carriers and/or excipients as is routine in the pharmaceutical art. For example, a suitable substance may be dissolved in physiological saline or water for injections. The exact nature of a formulation will depend upon several factors including the particular substance to be administered and the desired route of administration. In some cases the formulation will be one which is suitable for administration via the hepatic portal vein and/or via intraperitoneal injection or via other routes which help localize the inducer or agent to the liver. Suitable types of formulation are fully described in Remington's Pharmaceutical Sciences, Mack Publishing Company, Eastern Pennsylvania, 17^{th} Ed. 1985, the disclosure of which is included herein of its entirety by way of reference.

The inducers or agents may be administered by enteral or parenteral routes such as via oral, buccal, anal, pulmonary, intravenous, intra-arterial, intramuscular, intraperitoneal or other appropriate administration routes. In a preferred embodiment of the invention the inducer or agent will be administered intravenously. In some cases, the inducer or agent will be administered in such a way that it only reaches a localized region of the body of the subject, rather than the whole body. In one embodiment the inducer or agent will be specifically administered to the liver. In particular the inducer or agent will be administered via the hepatic portal vein. In other embodiments, and in particular where the inducer administered or generated is only capable of inducing apoptosis in hepatic stellate cells, but not any other cell type of the body, the inducer or agent will be administered via routes which cause exposure to a wider range of cell types such as intravenous administration.

A therapeutically effective amount of the inducer or agent is administered to the subject. The dose of inducer or agent may be determined according to various parameters, especially according to the substance used; the age, weight and condition of the patient to be treated; the route of administration; and the required regimen. A physician will be able to determine the required route of administration and dosage for any particular patient. In vitro and animal tests may be used to determine the likely effective does prior to administration to humans. A typical daily dose is from about 0.01 to 50 mg per kg of body weight, according to the activity of the specific inducer or agent, the age, weight and conditions of the subject to be treated, the type and severity of the degeneration and the frequency and route of administration. Preferably, daily dosage levels are from 5 mg to 2 g.

In the case of gliotoxin, or a derivative thereof, the subject may have, for example, from 0.1 to 20 mg gliotoxin per kg bodyweight, preferably from 1 to 10 mg gliotoxin per kg bodyweight, even more preferably from 1 to 5 mg gliotoxin per kg bodyweight administered. Similar dosage ranges may be employed for other inducers of the invention.

In some embodiments of the invention an agent comprising, or consisting essentially of, a nucleic acid molecule will be administered to the subject. This may be in any suitable form, such as in a virus, liposome, coated onto particles and/or as naked nucleic acid. Nucleic acid constructs may be administered by any available technique and/or route including any of those discussed above. In particular, the nucleic acid will be administered to the liver and preferably specifically delivered to hepatic stellate cells. Uptake of nucleic acid constructs may be enhanced by several known transfection techniques, for example those including the use of transfection agents. Examples of these agents includes cationic agents, for example, calcium phosphate and DEAE-Dextran and lipofectants, for example, lipofectam and transfectam. The dosage of the nucleic acid to be administered can be altered. Typically the nucleic acid is administered in the range of 1 pg to 1 mg, preferably to 1 pg to 10 μg nucleic acid for particle mediated gene delivery and 10 μg to 1 mg for other routes.

The inducer or agent can be administered prophylactically or to treat subjects who already have liver fibrosis. Thus in some cases the inducer or agent will be administered when the subject is known to have been exposed to an agent thought to promote liver fibrosis. Thus the subject may, for example, have just had a drug overdose or overdose of some other chemical known to cause liver damage. In other cases, the subject may actually have liver fibrosis and this may have developed to cirrhosis.

The inducer or agent may be administered in a single dose or in several doses such as one, two, three, five, ten or more doses. In the case where the inducer or agent is administered several times it may be given, for example, daily, every two days, at weekly intervals or monthly intervals. Treatment may be continued until the subject shows significant improvement in liver function and/or regression of liver fibrosis. Treatment may be at times when the individual is showing a marked increase in the level of fibrosis and/or has elevated exposure to the causative agent of the liver fibrosis.

Medicaments & Agents

The present invention also provides for the use of an inducer of hepatic stellate cell apoptosis, or of an agent capable of giving rise to an inducer of hepatic stellate cell apoptosis in vivo, in the manufacture of a medicament for treating liver disease in a subject, wherein the inducer or agent:
(a) can be selectively delivered to hepatic stellate cells in the liver of the subject;
(b) can selectively induce, or give rise to a selective inducer of, hepatic stellate cell apoptosis in the liver of the subject; and/or
(c) can generate the inducer specifically in hepatic stellate cells.

The present invention also provides for an agent for treating liver disease in a subject, the agent comprising an inducer of hepatic stellate cell apoptosis or an agent which can give rise to an inducer of hepatic stellate cell apoptosis, wherein the inducer or agent is:
(a) is selectively delivered to hepatic stellate cells in the liver of the subject;
(b) is selectively induces, or gives rise to a selective inducer, of hepatic stellate cell apoptosis in the liver of the subject; and/or
(c) can generate the inducer specifically in hepatic stellate cells.

In both of these embodiments of the invention, the inducer, agent, liver disease, subject to be treated and other aspects may be the same as in other embodiments of the invention.

The following Examples illustrate the invention.

### EXAMPLES

### Example 1

### Demonstration of the Selective Induction of Hepatic Stellate Cell Apoptosis by Gliotoxin and Regression of Liver Fibrosis in an In Vivo Model Following Administration of Gliotoxin

Materials & Methods

Reagents

Male Sprague-Dawley (200-225 g) rats were purchased from Charles River (Margate, Kent, England). Gliotoxin, bis-dethio-bis(methylthio)-gliotoxin (mt-glio), carbon tetrachloride (CCl₄), [m]-iodobenzylguanidine (m-IBG), and pyrrolidine dithiocarbamate (PDTC) were purchased from the Sigma Chemical Co., Dorset, England. Calcein-2AM, fluo-3AM, Caspase inhibitor 1 (Z-VAD-FMK), and quin-2-am were obtained from Calbiochem, Nottingham, England. Tetramethylrhodamine methylester (TMRM) was supplied by Molecular Probes, Eugene, Oreg. All other chemicals were of the highest purity available from local commercial sources.

Liver Cell Isolation, Culture, and Treatment

Rat hepatic stellate cells (HSCs) were isolated by pronase/collagenase perfusion and purified by isopycnic density centrifugation in Opti-prep™ (Nycomed, Amersham, England) and elutriation essentially as previously described (Bahr et al., Hepatology (1999) 29:839-848). Human hepatic stellate cells were isolated from discarded resected liver via a similar protocol (Trim et al., J. Biol. Chem., (2000) 275:6657-6663). The use of human liver tissue for scientific investigation was approved by the UK South and West Local Research Ethics Committee and was subject to patient consent.

Hepatic stellate cells were cultured as previously outlined (Bahr et al., supra and Trim et al., supra) for at least 14 days over which time they progressively increased the expression of a smooth muscle actin expression from undetectable levels at isolation (data not shown). It has been shown recently that liver myofibroblasts may contribute to liver fibrogenesis and that liver myofibroblasts may have the potential to contaminate passaged hepatic stellate cell cultures (Knittel et al., Gastronenterology, (1999) 117:1205-1221). In this study, only primary cultures of hepatic stellate cells were used, which were desmin and glial fibrillary acid protein positive (markers shown to be expressed in hepatic stellate cells, but not liver myofibroblasts—Knittel et al., supra).

Rat hepatocytes were isolated by collagenase perfusion essentially as previously described. (Harvey et al., Biochemical J., (1998) 331:273-281). Hepatocytes were cultured as outlined for hepatic stellate cells except that they were initially seeded onto collagen coated plates in William's medium E supplemented with 10% fetal calf serum and 1 μg/mL insulin for the first 2 hours.

Cells were treated with gliotoxin dissolved in DMSO vehicle (added to medium from a 2000-fold molar concentrated stock). Control cells received DMSO vehicle only (i.e., 0.05% vol/vol). All other additions to culture medium were made from concentrated stocks dissolved in DMSO or by direct addition to culture medium. Protein contents of wells were determined using the Lowry assay (Lowry et al., J. Biol. Chem., (1951)193: 265-275).

Determination of Caspase 3 Activity

Hepatic stellate cells cultured in 100 mm diameter dishes (Greiner, Frickenhausen, Germany) were harvested and pelleted by centrifugation. The medium supernatant was discarded and the cell pellet was washed in 1 mL of ice-cooled phosphate buffered saline (PBS). Caspase 3 (DEVDase) activity was determined using a colorimetric CaspACE kit (Promega, Southampton, England) using the manufacturer's instructions.

Examination of Low Molecular Weight DNA Fragmentation

Hepatic stellate cells cultured in 35-mm diameter dishes (Greiner) were harvested, pelleted by centrifugation and DNA fragmentation determined as outlined elsewhere (Elsharkawy et al., Hepatology (1999) 30:761-769).

Propidium Iodide Flow-Activated Cell Sorting Analysis

Detached cells were pelleted by centrifugation, washed in PBS, and resuspended in HYPO buffer (0.1% wt/vol) Na₃Citrate and 0.1% (wt/vol) Triton™ X-100 containing 50 mg/mL propidium iodide and incubated for 1 hour. The cells were then analyzed by fluorescence-activated cell sorter (FACS) with a Becton Dickinson (San Jose, Calif.) FACScan instrument using a 488 nm excitation wavelength with a band pass filter set at 530 nm. Attached cells were scraped in HYPO buffer after washing cells with PBS.

Terminal Deoxynucleotidyl Transferase Mediated Deoxyuridine Triphosphate Nick End Labeling Staining of Cultured Hepatic Stellate Cells and Histologic Sections

Rat and human hepatic stellate cells and rat liver tissue were fixed in 4% paraformaldehyde in PBS or 10% formalin in PBS, respectively, before being stained with Giemsa. DNA fragmentation was examined by labeling of 3′-OH DNA ends by the enzymatic addition of digoxygenin-labeled deoxyuridine triphosphate (dUTP) using terminal deoxynucleotidyl transferase using a kit from Boehringer essentially as described by the manufacturer. Rat liver tissue sections were pretreated with diethyl pyrocarbonate as described previously (Stahelin et al., J. Clin. Pathol. Mol. Pathol. (1998) 51:204-208) to reduce nonspecific reaction.

To determine if terminal deoxynucleotidyl transferase mediated deoxyuridine triphosphate nick end labeling (TUNEL) positive cells were activated hepatic stellate cells, TUNEL-positive nuclei were identified using 3-amino-9-ethylcarbazole staining (red) followed by immunostaining for α-smooth muscle actin with an alkaline phosphatase conjugated secondary antibody detected by fast blue essentially as previously described. (Iredale et al., J. Clin. Invest., (1998); 102:538-549).

Electromobility Shift Assay for NF-kB DNA Binding Activity

Crude high salt extractable nuclear protein was prepared from activated hepatic stellate cells for analysis of NF-kB DNA binding activity essentially as described (Elsharkawy et al., supra), aliquoted and stored at −80° C. until required. Extract protein concentrations were determined using the Lowry colorimetric assay (Lowry et al., supra) with bovine serum albumin as standard. A double stranded 5′ end labeled radiolabeled oligonucleotide [sense 5′-AGTTGAGGGGACTTTCCCAGGC (SEQ ID NO:1)] containing a consensus NF-kB DNA binding site as underlined (Baldwin et al., Annu. Rev. Immunol., (1996) 14:649-681) was used to determine NFkB DNA binding activity in crude nuclear extracts (Elsharkawy et al., supra).

Confocal Microscopy

Intracellular calcium concentrations were examined by preloading activated hepatic stellate cells seeded onto 35-mm diameter dishes with 2.5 μmol/L Fluo-3AM for two hours. Mitochondrial integrity was determined by coloading cells with 500 nmol/L TMRM and 1 μmol/M calcein-AM for three hours. After loading, the culture medium was removed and the cells were washed extensively with confocal buffer (145 mmol/L NaCl, 5 mml/L KCl, 1 mmol/L MgSO₄, 1 mmol/L NaH₂PO₄, 10 mmol/L HEPES, 25 mmol/L glucose, 1 mmol/L CaCl₂ and 2 mg/mL bovine serum albumin, pH 7.4) and incubated at 37° C. in 2 mL confocal buffer containing 1.5 μmol/L gliotoxin or DMSO vehicle control.

Fluorescence images were collected at 1 μm intervals using an Olympus BX50WI microscope fitted with the Biorad microradiance confocal scanning system. Fluo-3 and calcein fluorescence were excited at 488 nm and collected at 515-530 nm using a band width filter. TMRM was excited at 543 nm and collected at wavelengths greater than 570 nm.

Carbon Tetrachloride (In Vivo) Model of Liver Fibrosis

Rats were randomly sorted into groups and treated with 2 mL CCl₄:olive oil (1:1 [vol/vol])/kg body weight by intraperitoneal injection twice weekly to cause liver fibrosis. Control animals were treated with 1 mL olive oil/kg body weight by intraperitoneal injection. Gliotoxin was administered at up to 3 mg gliotoxin/kg body weight by intraperitoneal injection. Gliotoxin was dissolved in dimethyl sulfoxide (DMSO) as a vehicle, and control animals received DMSO alone.

At the required time, rats were killed by carbon dioxide asphyxiation and tissues removed for analysis. Serum was prepared and analyzed for alkaline phosphatase and alanine aminotransferase activities essentially as previously described. (Wright et al., Biochem. Pharmacol., (1992) 43:237-243) Histochemical staining of formalin-fixed liver sections with H&E, sirius red, and immunochemical staining for α-smooth muscle actin were performed essentially as previously described (Iredale et al., —1998—supra).

Results

Gliotoxin Stimulates the Apoptosis of Hepatic Stellate Cells In Vitro

Culture activated rat (14 day) and human (24 day) hepatic stellate cells were treated with DMSO solvent control or 1.5 μMol/L gliotoxin. Light microscopy of at least six separate preparations of cells showed that addition of gliotoxin resulted in striking morphologic alterations within one hour. Hepatic stellate cells changed from a flattened fibroblastic phenotype with distinct cell-cell interfaces to a substratum detached, rounded, and blebbed morphology. Within four hours of incubation, the morphologic alterations associated with gliotoxin treatment were observed in all hepatic stellate cells and a majority of the hepatic stellate cells had detached from the culture dish substratum.

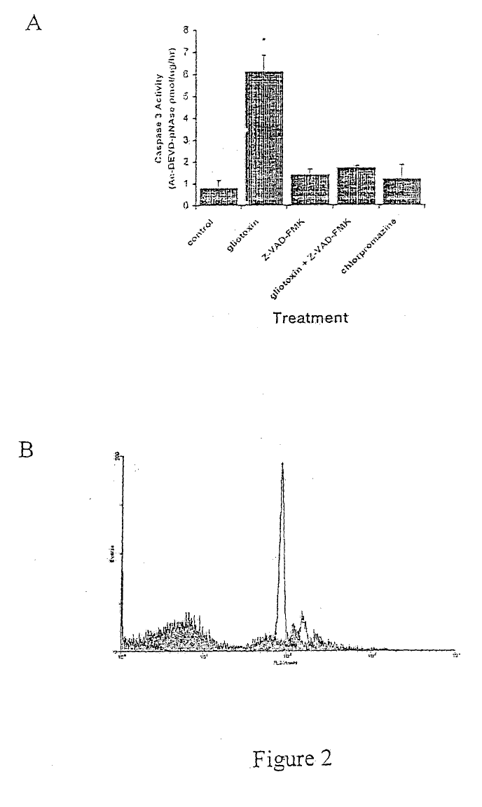

Caspase 3 (Ac-DEVD-pNA cleavage) activity was then examined in control and gliotoxin-treated hepatic stellate cells. Culture activated rat hepatic stellate cells (14 days) in 100 mm diameter plates were treated with 0.05% (vol/vol) DMSO, 1.5 μmol/L gliotoxin, 20 μmol/L Z-VAD-FMK or 200 μmol/L chlorpromazine for three hours. The cells were then harvested for examination of caspase 3 activity as outlined in the materials and methods sections. FIG. 2A shows the results obtained (the results shown are the mean and standard deviation of caspase activities determined from three separate experiments).

The results obtained show a significant (7.6-fold) increase in caspase 3 activity in gliotoxin-treated hepatic stellate cells after three hours that was inhibited by cotreatment of cells with the caspase inhibitor Z-VAD-FMK. The increase in caspase 3 activity observed with gliotoxin treatment was not seen when the hepatic stellate cells were treated with chlorpromazine. Chlorpromazine was toxic to hepatic stellate cells as judged by substratum detachment, morphologic alterations, and resulted in cells that were unable to exclude 0.1% (wt/vol) trypan blue. Gliotoxin treatment of hepatic stellate cells gave rise to detached cells that excluded 0.1% (wt/vol) trypan blue, suggesting that the cell membrane remains intact.

The effect of gliotoxin on the cleavage of DNA to a nucleosomal ladder was examined because this phenomenon is characteristic of apoptosis. Culture activated (14-day) rat hepatic stellate cells in six well plates were treated with either 0.05% (vol/vol) DMSO for four hours or 1.5 μmol/L gliotoxin for zero hours, half an hour, one hour, two hours or four hours . At each time point, both detached and attached cells were harvested together and low molecular weight DNA (20,000 g supernatant) fragmentation determined as outlined in the materials and methods section. At least three separate experiments were performed. 1 kilobase DNA ladder (Promega) was used as a molecular weight marker. The results obtained show that gliotoxin treatment of rat hepatic stellate cells results in a time dependent increase in DNA cleavage to a nucleosomal ladder with laddering beginning to be seen at two hours and substantial laddering visible at four hours.

The effect of various compounds on DNA laddering was then determined. Culture-activated (14-day) rat hepatic stellate cells in six-well plates were treated with:
(a) 0.05% (vol/vol) DMSO;
(b) 1.5 μmol/L mt-glio;
(c) 1.5 μmol/L gliotoxin;
(d) 1.5 μmol/L gliotoxin with 300 μmol/L pyrroline dithiocarbonate;
(e) 1.5 μmol/L gliotoxin with 100 μM Z-VAD-fMK;
(f) 1.5 μmol/L gliotoxin and 10 μM Z-VAD-fMK; or
(g) 1.5 μmol/L gliotoxin and 1 μM Z-VAD-fMK.
   After fours hours of treatment, DNA fragmentation was determined.

The generation of a nucleosomal ladder in response to gliotoxin treatment was inhibited completely at concentrations of 100 and 10 μm/L Z-VAD-fMk and substantial inhibition was still seen at concentrations as low as 1 μmol/L Z-VAD-FMK. However, concentrations as high as 100 μmol/L Z-VAD-FMK did not prevent the morphologic alterations caused by gliotoxin. These data suggest that caspase 3 induction and DNA cleavage to a nucleosomal ladder are late events in gliotoxin-stimulated apoptosis.

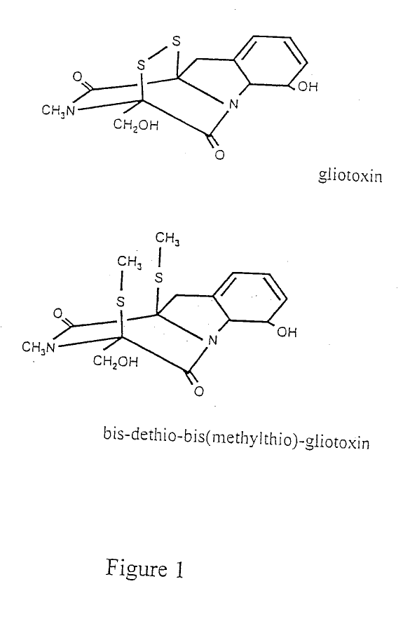

Interestingly, mt-glio (see FIG. 1 for the structure of mt-glio) had no effect on the morphology of rat and human hepatic stellate cells and did not result in the cleavage of DNA to a nucleosomal ladder. This suggests that the dithiol bridge in gliotoxin is essential for the ability to induce apoptosis and this is supported by the observation that the thiol-reducing agent pyrrolidine dithiocarbamate blocked the morphologic effects of gliotoxin in rat and human hepatic stellate cells and blocks the cleavage of DNA to a nucleosomal ladder. The oxidation of a critical thiol(s) by gliotoxin is therefore an event that occurs before morphologic and DNA cleavage events.

To characterize the extent and significance of apoptosis in response to gliotoxin, rat hepatic stellate cell DNA strand breaks were assessed by FACS analysis of propidium iodide stained cells. Culture-activated (14-day) rat hepatic stellate cells were treated with 0.05% (vol/vol) DMSO vehicle control or with 1.5 μmol/L gliotoxin for two hours, harvested, and stained with propidium iodide as outlined in the materials and methods section. Before staining, both control and gliotoxin-treated hepatic stellate cells were found to exclude trypan blue indicating that the cell membranes were intact. The cells were then analyzed by FACs and the results obtained are shown in FIG. 2B.

FIG. 2B shows the events of control cells (1×10⁴; clear) compared with events from gliotoxin-treated hepatic stellate cells (1×10⁴; shaded). The results are typical of six separate experiments. Control hepatic stellate cell propidum iodide staining resulted in a discrete peak in nuclei fluorescence derived from viable stellate cells containing undegraded DNA. In addition, a smaller peak of greater fluorescence intensity in the control is likely to be nuclei derived from stellate cells that were undergoing mitosis. Treatment with gliotoxin gave hepatic stellate cells that excluded trypan blue, but the treated cells give rise to a broad low level of fluorescence when their nuclei were stained with propidium iodide. This indicates DNA cleavage.

DNA strand breaks were also characterized by TUNEL staining. Culture-activated (14-day) rat hepatic stellate cells were treated with 0.05% (vol/vol) DMSO or 1.5 μmol/L gliotoxin for two hours and DNA strand breaks examined by TUNEL staining as outlined in Materials and Methods. TUNEL staining fidelity was determined by staining control and gliotoxin-treated cells without the incorporation of dUTP in the protocol and resulted in staining similar to that for hepatic stellate cells treated with the DMSO control. Gliotoxin treatment of rat hepatic stellate cells gave rise to extensive TUNEL-positive staining in contrast to control cells.

The addition of gliotoxin to activated human hepatic stellate cells in vitro resulted in similar morphologic alterations to that observed with rat hepatic stellate cells. In general, human hepatic stellate underwent morphologic alterations more rapidly than rat hepatic stellate cells, although human hepatic stellate cells did not cleave their DNA to oligonucleosomal-length fragments (six independent experiments were carried out to confirm this). However, DNA cleavage was detected in response to gliotoxin treatment when human hepatic stellate cell nuclei were stained with propidium iodide or by TUNEL staining, suggesting that gliotoxin initiates the apoptosis of human as well as rat hepatic stellate cells.

Gliotoxin Kills Rat Hepatocytes Only at High Concentrations and Via a Necrotic Mechanism of Cell Death

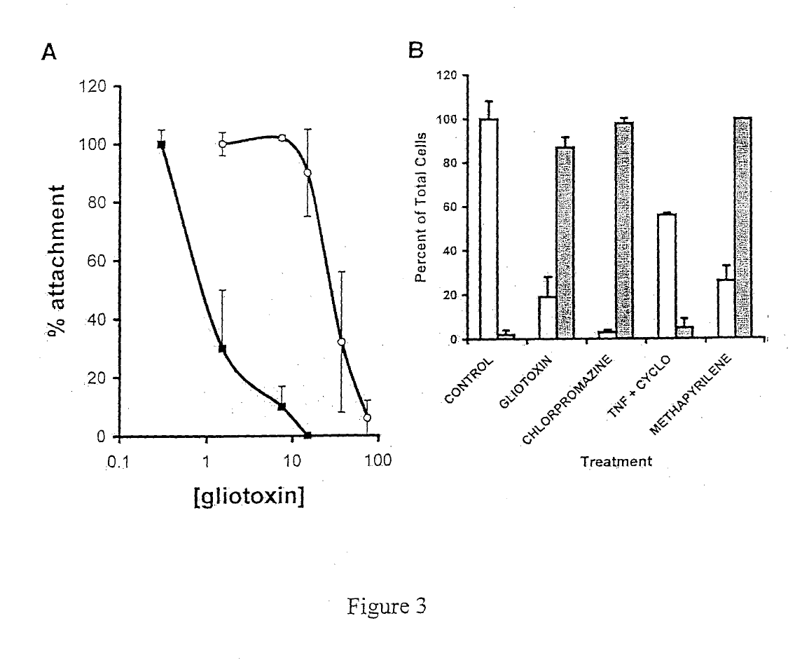

The necessary doses of gliotoxin required to kill rat hepatic stellate cells and hepatocytes were compared. Culture-activated (14-day) rat hepatic stellate cells or rat hepatocytes were treated with either 0.05% (vol/vol) DMSO or 1.5 μmol/L gliotoxin and cell attachment determined by direct assay of protein in each well after four hours as outlined in Materials and Methods. The results obtained are depicted in FIG. 3A and are expressed as the mean and standard deviation percentage attachment versus DMSO control for three separate experiments. The results obtained show that significantly (10 to 100-fold) higher concentrations of gliotoxin were required to kill rat hepatocytes in comparison to rat hepatic stellate cells in vitro. Longer incubation of gliotoxin with hepatocytes did not result in significantly different levels of cell death.

The effect of various compounds on cell viability and attachment was determined. Viability of rat hepatocytes as judged by attachment and 0.1% (wt/vol) trypan blue exclusion after four hours treatment with:
(a) 0.05% (vol/vol) DMSO;
(b) 50 μmol/L gliotoxin;
(c) 200 μmol/L chlorpromazine;
(d) 10 ng/mL TNF-α with 10 μmol/L cycloheximide; or
(e) 200 μmol/L methapyrilene.
   was determined.

The results obtained are depicted in FIG. 3B. These show that treatment with 50 μmol/L gliotoxin causes significant reductions in cell attachment to the substratum and that approximately 90% of hepatocytes do not exclude 0.1% (wt/vol) trypan blue, a level similar to that caused by hepatotoxins chlorpromazine and methapyrilene (Ratra et al., Toxicology (1998)130: 79-93). In contrast, the treatment of hepatocytes with tumor necrosis factor alpha (TNF-α) and cycloheximide (which is known to stimulate hepatocyte apoptosis—Bradham Mol. Cell. Biol., (1998) 18: 6353-6364) resulted in cellular detachment without a loss of membrane integrity as judged by 0.1% (wt/vol) trypan blue exclusion.

DNA laddering was also used to compare the effect of gliotoxin, chlorpromazine, methapyrilene and also the combination of TNF-α with cycloheximide on hepatocytes and hepatic stellate cells. Rat hepatocytes were treated for four hours with:
(a) 0.05% (vol/vol) DMSO;
(b) 50 μmol/L gliotoxin;
(c) 200 μmol/L chlorpromazine
(d) 10 ng/mL TNF-α with 10 μmol/L cycloheximide; or
(e) 200 μmol/L methapyrilene.
   Following treatment, DNA laddering was assessed. The results obtained showed that only hepatocytes treated with TNF-α and cycloheximide cleaved their DNA to a nucleosomal ladder in contrast with hepatocytes treated with either gliotoxin, chlorpromazine, or methapyrilene.

Interestingly, DNA cleavage was induced in hepatic stellate cells in a concentration-dependent manner and apoptosis, as judged by this criterion, was detectable in hepatic stellate cells treated with concentrations as low as 300 nmol/L gliotoxin. However, at relatively high concentrations of gliotoxin (37.5 μmol/L) there was a reduction in the level of DNA cleavage detected when compared with hepatic stellate cells treated with lower concentrations of gliotoxin. This suggests that gliotoxin was necrotic to both hepatic stellate cells and hepatocytes at high concentrations, but that gliotoxin stimulated apoptosis only in hepatic stellate cells at low concentrations.

The Mechanism of Action of Gliotoxin: Role of NF-kB, the Mitochondrial Permeabilty Transition, and Calcium in Gliotoxin-Dependent Apoptosis of Hepatic Stellate Cells

The effect of gliotoxin on NF-kB activity in hepatic stellate cells was investigated. Culture-activated (14-day) rat hepatic stellate cells were incubated in the presence or absence of 10 ng/mL TNF-α fifteen minutes after addition of:
(a) 0.05% (vol/vol) DMSO vehicle;
(b) 1.5 μmol/L gliotoxin;
(c) 5 mM N-acetyl cysteine;
(d) 50 μM calpain inhibitor 1;
(e) 0.5 μm dexamethasone; or
(f) 3.6 mM pentoxyfilline.

After a further 30 minutes, and before any significant morphologic changes, cells were washed with ice-cooled PBS, nuclear extracts prepared, and NF-kB DNA binding activity assessed by gel shift analysis. As a further control nuclear extract containing 50-fold molar excess cold NF-kB oligonucleotide was run to demonstrate specific saturable binding. Each lane contained 6 mg of protein and the results were the same for at least three separate experiments.

The results obtained show that gliotoxin does not have a marked inhibitory effect on constitutive NF-kB DNA binding activity, but that it inhibits TNF-α-inducible NF-kB DNA binding activity in activated rat hepatic stellate cells. Other reported inhibitors of NF-kB DNA binding activity: N-acetyl cysteine, (Staal et al., Proc. Natl. Acad. Sci. USA (1990) 87:9943-9947); pentoxyfilline (Lee et al., Am. J. Physiol., (1997) 273:G1094-G1100); and dexamethasone (Caldenhoven et al., Mol. Endocrinol., (1995) 9:401-412) had little effect on either constitutive or TNF-α-induced activated rat stellate cell NF-kB DNA binding activity.

Calpain inhibitor 1 has been reported to inhibit NF-kB by a similar mechanism to gliotoxin, via inhibition of 1 kB degradation (Palombella et al., Cell (1994) 78:773-785). The results showed that calpain inhibtor 1 treatment inhibited the formation and altered the mobilities of both constitutive and TNF-α-inducible NF-kB DNA binding complexes detected by gel shifts. In addition, CI-1 treatment resulted in the appearance of a low mobility complex that was not present in control (CI-1-free) stellate cell nuclear extracts. However, despite these effects on NF-kB DNA binding activity, CI-1 treatment did not result in the apoptosis of rat hepatic stellate cells as judged by morphologic criteria and biochemical criteria such as induction of caspase 3 activity or cleavage of DNA to a nucleosomal ladder.

Calcein and TMRM are fluorescent dyes that accumulate into the cytoplasm and mitochondria, respectively. They can be used to visualize the onset of the mitochondrial permeability transition (Bradham et al., supra). The mitochondrial permeability transition (MPT) results in an abrupt increase in the permeability of the inner mitochondrion membrane and is implicated in the release of cytochrome C, caspase activation, and apoptosis (Yang et al., Science (1997) 275: 1129-1132).

Culture activated rat hepatic cells (14 day) were loaded with calcein (green fluorescence) and TMRM (red fluorescence) and then treated with either 0.05% (vol/vol) DMSO vehicle or alternatively 1.5 μmol/L gliotoxin. Green fluorescence and red fluorescence were imaged at regular time points as outlined in Materials and Methods and the results obtained were typical for at least three separate experiments.

Loading of rat hepatic stellate cells with calcein and TMRM resulted in separate cellular distribution, indicating that mitochondria were polarized and impermeable to low molecular weight solutes in untreated cells. Addition of gliotoxin to activated rat hepatic stellate cells resulted in no apparent effect until after two hours of incubation when there was initially a migration of TMRM fluorescence to the periphery of cells followed by a loss of TMRM fluorescence at four hours. The loss of TMRM fluorescence intensity is likely associated with a loss of mitochondrial permeability (Bradham et al., supra). In all experiments, however, mitochondrial migration and changes in membrane permeability occurred after significant morphologic alterations, suggesting that the MPT is a late event in gliotoxin dependent apoptosis.

DNA laddering was then studied to see if it supported the fluorescence imaging results. Culture activated (14 day) rat hepatic stellate cells in six well plates were treated with:
(a) 0.05% (vol/vol) DMSO;
(b) 1.5 μmol/L gliotoxin;
(c) 1.5 μmol/L gliotoxin with 100 μmol/L tamoxifen;
(d) 1.5 μmol/L gliotoxin with 1 mg/ml actinomycin d;
(e) 1.5 μmol/L gliotoxin with 10 μmol/L cycloheximide;
(f) 1.5 μmol/L gliotoxin with 10 μmol/L cyclosporin;
(g) 1.5 μmol/L gliotoxin with 250 μmol/L m-iodobenzylguanidine;
(h) 1.5 μmol/L gliotoxin with 50 μmol/L quin-2-am;
(i) 50 μmol/L guin-2-am alone;
(j) 250 μmol/L m-iodobenzylguanidine;
(k) 10 μmol/L cyclosporin;
(l) 10 μmol/L cycloheximide;
(m) 1 μg/ml actinomycin d; or
(n) 100 μmol/L tamoxifen.

After four hours of treatment, DNA fragmentation was determined as outlined in Materials and Methods. The results obtained were typical of at least three separate experiments. Note that in all cases in which hepatic stellate cells were treated with gliotoxin irrespective of other additions to the medium, cellular detachment and other morphologic alterations still occurred.

Inhibitors of MPT, m-iodobenzylguanidine (Juedes et al., FEBS. Lett., (1992) 313:39-42) and tamoxifen (Custodio et al., Toxicol. Appl. Pharmacol., (1998) 152: 10-17) but not cyclosporin A prevented the cleavage of DNA to a nucleosomal ladder, but did not prevent the detachment of hepatic stellate cells from the substratum and other morphologic alterations caused by gliotoxin. The MPT is therefore likely to be upstream of DNA cleavage to a nucleosomal ladder, but downstream of early events such as NF-kB inhibition.

MPT results in futile Ca²⁺ cycling by mitochondria, which enhances the likelihood of cell death (Crompton et al., Biochem. J. (1999) 341: 233-249). The role of the MPT in the late events of gliotoxin apoptosis was also therefore assessed by measuring intracellular calcium levels. Culture-activated (14-day) rat hepatic stellate cells in six-well plates were loaded with fluo-3 and fluorescence imaged as outlined in the Materials and methods section after treatment with DMSO control or 1.5 μmol/L gliotoxin. The results showed that intracellular Ca²⁺ levels do not rise until two hours of incubation with gliotoxin and after cell detachment. The cell-permeable Ca²⁺ chelator quin-2AM also blocks DNA cleavage without preventing cell detachment.

The Effect of Gliotoxin Treatment in a Rat Model of Liver Fibrosis

Treatment of rats with carbon tetrachloride for seven weeks resulted in a significant increase in serum alanine aminotransferase activity, but not of serum alkaline phosphatase activity, suggesting that carbon tetrachloride has primarily damaged parenchymal liver cells (see Table 1).

A pilot study for gliotoxin toxicity suggested that gliotoxin at a dose of 3 mg/kg body weight did not cause any apparent ill effects in rats and there was no evidence of hepatic damage on examination of histologic sections of the liver. A single injection of gliotoxin to control or carbon tetrachloride treated rats did not result in any significant change in serum liver enzyme levels supporting evidence that gliotoxin was not hepatotoxic at this dose alone and did not modulate the hepatotoxicity of carbon tetrachloride (see Table 1).

TABLE 1
Effect of Gliotoxin Treatment in an In Vivo Model of Liver Fibrosis

| | Gliotoxin | | Single gliotoxin injection | | | | Weekly gliotoxin injection | | | |
|---|---|---|---|---|---|---|---|---|---|---|
| | treatment | | | | | Sirius red | | | | Sirius red |
| CCl₄ | (mg | | | | Liver | staining (mid | | | Liver | staining (mid |
| treat- | gliotoxin/kg | | Serum ALP | Serum ALT | α-sma | intralobular | Serum ALP | Serum ALT | α-sma | intralobular |
| ment* | body wt*) | | (μ/mL) | (μ/mL) | staining | μm^{§}) | (μ/mL) | (μ/mL) | staining^{¶} | μm^{§}) |
| − | − | | 319 ± 44 | 81 ± 30 | 1.9 ± 0.54^{†} | 0 | 342 ± 13 | 89 ± 5 | 1.7 ± 2.03 | 0 |
| − | 0.3 | mg/kg | 336 ± 48 | 59 ± 4 | 2.5 ± 0.70^{†} | 0 | n/d | n/d | n/d | n/d |
| − | 3 | mg/kg | 269 ± 25 | 50 ± 5 | 1.2 ± 0.24^{†} | 0 | 249 ± 26 | 89 ± 58 | 1.4 ± 1.38 | 0 |
| + | − | | 628 ± 107 | 1020 ± 476 | 37 ± 14.8 | 12 ± 3.6 | 709 ± 146 | 1900 ± 758 | 29 ± 6 | 16 ± 8.0 |
| + | 0.3 | mg/kg | 790 ± 128 | 1310 ± 342 | 35 ± 11.4 | 9 ± 2.3 | n/d | n/d | n/d | n/d |
| + | 3 | mg/kg | 755 ± 295 | 1740 ± 1100 | 16 ± 6.4^{†} | 7 ± 3.6^{£} | 429 ± 25^{a} | 300 ± 111^{b} | 6 ± 1.2^{†} | 4.6 ± 3.1^{£} |

*CCl₄ was administered twice weekly for seven weeks (single gliotoxin injection) or 4 weeks (weekly gliotoxin injection) by intraperitoneal injection mixed 1:1 (vol/vol) with olive oil, and controls received olive oil only. Between 3-5 animals were in each treatment group.
^{#}Gliotoxin was dissolved in DMSO, and controls received DMSO alone. Animals were killed 48 hours after the last CCl₄ injection (24 hours after a single/final injection of gliotoxin).
^{a,b}Significantly different (P > 95%) serum liver enzyme levels vs. CCl₄ - treated only animals
^{¶}Mean and SD of the number of α-sma-positive cells as determined by immunohistochemical staining. A slide for each animal was strained and the mean count of 20 randomly selected high power fields (×110) was used to calculate a group mean and SD.
^{†}Significantly different (P > 95%) mean number of α-smooth muscle actin positive cells per 20 randomly selected fields compared with CCl₄-treated only rats using the Student t test (2 tailed)
^{§}The mid-intraobular width of sirius red-stained bands (mean of 10 individual measurements/animal).
^{£}Significantly lower mean width of sirius red stained band compared with CCl₄-treated only rats using the Student t test (1 tailed).
n/d not determined

The effect of a single injection of gliotoxin on α-smooth muscle actin liver immunostaining after treatment for seven weeks with carbon tetrachloride was then determined. One day after the final injection of carbon tetrachloride, rats were administered gliotoxin and killed after a further day.

Liver sections were stained from rats treated with:
(a) vehicle (olive oil) for seven weeks followed by DMSO;
(b) vehicle (olive oil) for seven weeks followed by 3 mg gliotoxin/kg body weight;
(c) carbon tetrachloride for seven weeks followed by DMSO; and
(d) carbon tetrachloride for seven weeks followed by 3 mg gliotoxin/kg body weight.
   The results obtained were typical for five animals per treatment.

From the stained liver sections it could be seen that carbon tetrachloride treatment resulted in significant hepatocellular fatty change and in the proliferation of α-smooth muscle actin-positive hepatic stellate cells in histologic sections of liver. The results also showed that a single gliotoxin injection to carbon tetrachloride-treated rats has a pronounced effect on the characteristics and intensity of α-smooth muscle actin immunostaining. Quantitative examination of sections from the animals of each treatment group indicated that the number of α-smooth muscle actin-positive cells in carbon tetrachloride treated rat liver was reduced by 57% through the administration of a single dose of 3 mg/kg body wt of gliotoxin (see Table 1).

The effect of a single injection of gliotoxin on liver TUNEL staining after treatment for seven weeks with carbon tetrachloride was measured. One day after the final injection of carbon tetrachloride, rats were administered gliotoxin and then killed after a further day. Control animals received DMSO alone in place of gliotoxin. Liver sections from the rats were TUNEL stained with or without the incorporation of dUTP in the staining protocol. Sections were then counterstained with hematoxylin.

The TUNEL staining of histologic sections indicated that there was an increase in the number of TUNEL-positive cells in gliotoxin treated rat liver in regions staining for α-smooth muscle actin. Dual staining for TUNEL and α-smooth muscle actin indicated colocalization, confirming that observed reduction in numbers of activated hepatic stellate cells in fibrotic liver in response to gliotoxin was mediated via apoptosis.

The effect of a single injection of gliotoxin on liver sirius red staining after treatment for seven weeks with carbon tetrachloride was then determined. As before, one day after the final injection of carbon tetrachloride, rats were administered gliotoxin and killed after a further day.

Stainings were performed on liver sections from rats treated with:
(a) vehicle (olive oil) for seven weeks followed by DMSO;
(b) vehicle (olive oil) for seven weeks followed by 3 mg gliotoxin/kg body weight;
(c) carbon tetrachloride for seven weeks followed by DMSO; and
(d) carbon tetrachloride for seven weeks followed by 3 mg gliotoxin/kg body weight.
   Results were observed to be equivalent in five separate animals subjected to each treatment.

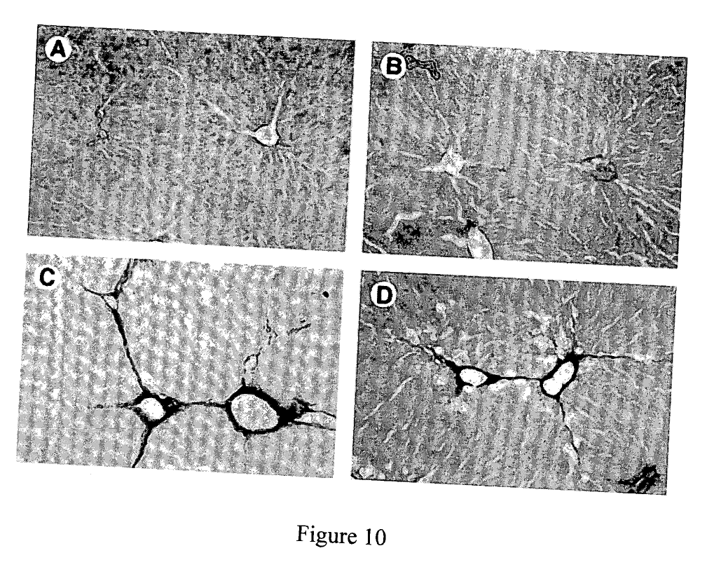

The blinded examination of sirius red stained liver sections to identify collagens indicated that a single injection of gliotoxin at 3 mg/kg significantly reduced fibrosis. The results for the stainings (a) to (d) are shown in FIG. 10. The mean intralobular thickness of fibrotic bands was measured under high power using an eye-piece graticule. Table 1 shows that the mean intralobular thickness of fibrotic bands was significantly reduced in liver sections from carbon tetrachloride treated rats also treated with gliotoxin compared with rats treated only with carbon tetrachloride.

The results obtained indicate that it is possible to promote resolution of liver fibrosis by stimulating hepatic stellate cell apoptosis with gliotoxin. Table 1 indicates that it may also be possible to modulate fibrogenesis through the administration of gliotoxin during the liver insult, because gliotoxin administration also significantly reduces the number of activated hepatic stellate cells and thickness of fibrotic bands in rats treated with carbon tetrachloride. There is no evidence that long term administration of gliotoxin is itself hepatotoxic in agreement with the in vitro studies conducted here. Indeed, gliotoxin administration significantly reduces the levels of liver serum enzymes caused by carbon tetrachloride treatment (see Table 1), suggesting that an inhibition of fibrogenesis may protect against hepatic necrosis.

Discussion

The data presented here demonstrate unequivocally that gliotoxin stimulates the immediate and complete apoptosis of culture activated hepatic stellate cells isolated from both rat and human liver. Moreover, the data indicates that gliotoxin is also effective in mediating hepatic stellate cell apoptosis in vivo after the development of fibrosis.

Hepatic stellate cells are known to secrete some of the factors involved in resolution of liver fibrosis such as, for example, particular matrix metalloproteases involved in the breakdown of fibrotic matrix. Although apoptosis of hepatic stellate cells occurs in the natural resolution of liver fibrosis, it might well have been expected that the simultaneous elimination of the cells mediating a wound healing response in the liver, as opposed to a staged reduction, would profoundly disturbed hepatic structure and function rather than promote resolution of liver fibrosis. Furthermore, the liver must have a finite capacity for the clearance of apoptotic hepatic stellate cells and hence it might have been expected that the induction of stellate cell apoptosis could have caused the number of apoptotic stellate cells to have exceeded this capacity. If this occurred, then the apoptotic cells which were not successfully removed could have caused secondary necrosis. Thus the experimental results obtained here show for the first time that induction of stellate cell apoptosis can successfully promote the resolution of liver fibrosis in vivo without adverse consequences to the hepatic phenotype.

Evidence presented here indicates that gliotoxin may mediate its effects through alternative or additional mechanisms to the NF-kB pathway. In activated hepatic stellate cells, gliotoxin did not strongly inhibit NF-kB DNA binding activity, in contrast to an inhibition observed in quiescent hepatic stellate cells or TNF α-treated activated rat hepatic stellate cells. Indeed, calpain inhibitor 1 failed to stimulate the apoptosis of rat hepatic stellate cells, yet modulated the DNA binding activity of NF-kB, and the thiol reductant PDTC (also reported to inhibit NF-kB (Schreck et al., J. Exp. Med. (1992) 175:1181-1194) protected hepatic stellate cells from both the morphologic and apoptotic effects of gliotoxin. Nevertheless, the effects of gliotoxin in vivo may act directly on NF-kB in a functionally meaningful way because in the presence of liver inflammation, sinusoidal TNF-α concentration may be raised.

It is likely that cellular targets other than NF-kB are critical to the mechanism of gliotoxin-dependent apoptosis. A critical cellular target of gliotoxin may be the mitochondrial permeability transition (MPT). Thiols have been reported to play a functional role in the regulation of the MPT (Crompton et al., Biochem. J., (1999) 341: 233-249) and gliotoxin, through its disulfide bridge (FIG. 1), is known to covalently react with protein thiols (Waring et al., Biochem. Pharmacol., (1995) 49:1195-1201).

The MPT is constituted by a complex of proteins including the voltage dependent anion channel, the adenine nucleotide translocase and cyclophilin D. Under certain conditions (e.g., oxidative stress, high mitochondrial Ca²⁺ and inorganic phosphate levels) these form a pore at contact sites between the inner and outer mitochondrial membranes that permits the efflux of molecules less than 1.5 kilodaltons from the matrix (Crompton et al., supra). Opening of the MPT is implicated in both necrotic and apoptotic cell death (Crompton et al., Supra). Gliotoxin has been shown to stimulate the release of Ca²⁺ from rat skeletal and liver mitochondria (Schweizer et al., Biochemistry (1994) 33:13401-13405 and Silva et al., Redox. Rep., (1997) 3:331-341) and therefore the ability of the Ca²⁺ chelator quin-2-am and inhibitors of the MPT (m-IBG and tamoxifen) to block DNA cleavage to a nucleosomal ladder suggests that the MPT and mitochondrial Ca²⁺ play a pivotal role in at least the late stages of apoptosis in response to gliotoxin. The inability of CsA to inhibit DNA cleavage to a nucleosomal ladder indicates that gliotoxin may form direct mixed disulfide with protein(s) that constitute the MPT pore such as cyclophilin D, thereby preventing CsA binding. The MPT has been shown to regulate caspase activation through its involvement in cytochrome c release, (Yang et al., supra) and the ability of the caspase inhibitor Z-VAD-FMK to block caspase 3 induction and DNA cleavage to a nucleosomal ladder in gliotoxin-treated rat hepatic stellate cells indicates that caspases are regulating DNA cleavage. However, higher concentrations of Z-VAD-FMK failed to block the morphologic alterations caused by gliotoxin, suggesting that there are caspase independent (and possibly MPT-independent) effects of gliotoxin in hepatic stellate cells.

It is of major importance to determine whether enhanced apoptosis of hepatic stellate cells promotes remodeling of the fibrotic liver. The studies here have shown that by increasing the rate of hepatic stellate cell apoptosis, the fibrotic bands in gliotoxin treated animals become attenuated. The rapidity with which this occurs (within 24 hours) suggests that the potential for rapid collagen turnover exists in the fibrotic liver. Additionally, gliotoxin may have inhibited further net collagen synthesis by inhibiting collagen accumulation. The effects of a single injection of gliotoxin 24 hours after administration were determined (to reduce any systemic effects of gliotoxin) and demonstrated significant enhancement in recovery from liver fibrosis. With long term treatment with gliotoxin (i.e., during hepatic insult), even more significant effects on liver fibrosis were observed.

The data presented here indicates that gliotoxin will effectively induce hepatic stellate cell apoptosis. In addition, it has been demonstrated that induction of apoptosis in hepatic stellate cells enhances the resolution of experimental fibrosis. Taken together, these results show that a strategy based on inducing hepatic stellate cell apoptosis is likely to prove an effective antifibrotic approach.

### Example 2

### Demonstration that Apoptosis of Hepatic Stellate Cell Apoptosis is Inhibited by the Action of TIMPs

Materials & Methods

Isolation of Human and Rat Hepatic Stellate Cells

Human hepatic stellate cells were extracted from the margins of normal human liver resected for colonic metastatic disease as previously described (Iredale et al., Clin. Sci., (1995) 89: 75-81). Rat hepatic stellate cells were extracted from normal rat liver by Pronase and collagenase digestion and purified by centrifugal elutriation as described (Arthur et al., J. Clin. Invest., (1989) 84:1076-1085). Extracted hepatic stellate cells were cultured on plastic until they were activated to a myofibroblastic phenotype after 7 to 10 days. Human and rat hepatic stellate cells were used for experiments after activation in primary culture or before fourth passage. Cells were cultured in Dulbecco's modified Eagle's medium in the presence of 16% fetal calf serum and antibiotics.

Effect of TIMP-1 on Hepatic Stellate Cell Proliferation

Hepatic stellate cells were cultured in 24-well tissue culture plates. These were washed with serum-free medium for 24 h, and then the cells were exposed to TIMP-1 at a concentration range of 1-100 ng/ml for 24 h and then pulsed with tritiated thymidine (0.5 μCi/well) for 18 hours before scintillation counting as previously described (Boulton et al., Clin. Sci., (1995) 88: 119-130).

Stimulation of HSC Apoptosis and Examination of Nuclear Morphology by Acridine Orange

Apoptosis of hepatic stellate cells was induced by absolute serum deprivation, cycloheximide treatment (Issa et al., Gut (2001) 48: S48-S57), or exposure to nerve growth factor as previously described (Trim et al., Am. J. Pathol., (2000) 156, 1235-1243). Hepatic stellate cells were cultured in 24-well tissue culture plates. Rat and human hepatic stellate cells were exposed to proapoptotic stimuli with and without recombinant TIMP-1 (Biogenesis, Poole, UK) and other manipulations as detailed below. Following a 4 hour incubation at 37° C., nuclear morphology was assessed by adding acridine orange to each well (final concentration 1 μg/ml) and observing the cells under blue fluorescence. The total number of apoptotic bodies was counted, and any apoptotic bodies floating in the supernatant were included by racking up the objective lens. The total number of cells per field was counted, and an apoptotic index was calculated. Each condition was performed in duplicate, and three high power fields were counted for each well. Experiments were repeated in parallel following an 18 hour incubation in serum-free conditions. To examine for autocrine effects, hepatic stellate cells were incubated for 18 hours with azide-free polyclonal neutralizing antibodies to TIMP-1 and a nonimmune IgG control (Santa Cruz Biotechnology, Inc., Santa Cruz, Calif.), and responses were assessed by acridine orange staining and counting. Parallel experiments using the nonfunctional T2G mutant N-TIMP-1 and wild type TIMP-1 proteins were performed in which apoptosis was induced by cycloheximide and assessed by the acridine orange technique.

TUNEL Staining

Hepatic stellate cells were cultured on glass chamber slides and then exposed to 50 μM cycloheximide for 18 h with and without TIMP-1 (100 ng/ml). Slides were then stained for DNA fragmentation characteristic of apoptosis by the TUNEL reaction as previously described (Iredale et al., J. Clin. Invest., (1998) 102, 538-549) with the modifications recently described to reduce false positivity (Stahelin et al., Mol. Pathol., (1998) 51, 204-208). Each slide was then analyzed by a blinded observer who counted the number of TUNEL-positive apoptotic figures and the TUNEL-negative cells over 10 high power fields for each condition.

Determination of Caspase-3 Activity

To support the data from acridine orange counting and TUNEL staining, experiments with recombinant TIMP-1, the inactive T2G mutant N-TIMP-1, the wild type TIMP-1 proteins, and the broad spectrum caspase inhibitor benzyloxycarbonyl-Val-Ala-Asp-fluoromethylketone were repeated. Apoptosis was quantified by a colorimetric assay for caspase-3 activity (Promega) according to the manufacturer's instructions. To determine whether TIMP-1 directly inhibited apoptosis of caspase-3, each recombinant protein was incubated with recombinant caspase-3 for 1 hour before adding the caspase-3 substrate, and then caspase-3 activity was measured as described above.

Measurement of DNA Concentration by PicoGreen Fluorescence

Cultured hepatic stellate cells were harvested with a sterile cell scraper, pelleted by centrifugation, and then resuspended in 500 μl of TE buffer (10 mmol/Tris-HCl, 1 mmol/liter EDTA, pH 8.0) before sonication for 15 min. 100 μl of PicoGreen (Molecular Probes, Inc., Eugene, Oreg.) at 1:200 dilution was added to 100 μl of sample and incubated in the dark at room temperature for 5 min. Standards were made from herring sperm DNA. Fluorescence was measured using a Cytofluor II Microwell Fluorescence reader (Perceptive Biosystems, Framingham, Mass.) at standard wavelengths (excitation 485 nm, emission 530 nm). Concentrations of double-stranded DNA in the samples were calculated from the standard curve.

Western Blotting for Smooth Muscle Actin and Bcl-2

Western blot analysis of rat liver tissue and hepatic stellate cells was undertaken using a monoclonal anti-α smooth muscle action antibody (Sigma) and a monoclonal antibody to Bcl-2 (Santa Cruz Biotechnology) to detect protein expression. The extracted proteins were subjected to electrophoresis on 12% SDS-PAGE gel after normalization for protein content. After resolution, the protein samples were electrotransferred onto polyvinylidene difluoride. The membrane was blocked for 1 hour in 5% nonfat dry milk in TBS. Membranes were incubated overnight at room temperature with the primary antibody (1:500) or with nonimmune IgG (as negative control) in TBS. Membranes were washed three times for 15 min in 0.1% Tween TBS (TTBS) before the addition of the secondary antibody (rabbit anti-mouse IgG horseradish peroxidase in a 1:2000 dilution) in TBS containing 0.5% nonfat dry milk for 1 h. The membranes were then washed in TTBS twice for 10 min, followed by distilled water for 10 min. Reactive bands were identified using ECL (Amersham Biosciences) and autoradiography according to the manufacturer's instructions.

Experimental Models of Progressive Fibrosis and Fibrosis Recovery

Experimental models of reversible fibrosis and cirrhosis were established by injecting cohorts of 12 Sprague-Dawley rats with carbon tetrachloride twice weekly intraperitoneally for 6 and 12 weeks, respectively. For each model, livers were harvested at peak fibrosis (immediately after the final injection of carbon tetrachloride) and at 5 and 15 days of spontaneous recovery (n=4 at each time point in each model). Harvested livers were split and fixed for hematoxylin and eosin and Sirius Red staining, and a portion was snap frozen for biochemical and molecular analysis. Histological analysis of each liver was undertaken, and in addition samples of frozen liver at peak fibrosis and 15 days of recovery were analyzed for hydroxyproline and total collagenase activity as previously described (Iredale et al., J. Clin. Invest., (1998) 102: 538-549). The MMPs that would be expected to show activity in this assay are the interstitial collagenases (MMP-1 and MMP-13), gelatinase A (MMP-2), and membrane type 1 MMP (MMP-14). Further sections were cut from each liver, deparaffinized, and subjected to microwave antigen retrieval before being immunostained for smooth muscle actin exactly as previously described (Iredale et al., (1998) supra). Three normal untreated rat livers were also harvested for use as controls in individual experiments. The number of smooth muscle actin positive cells was counted by a blinded observer exactly as described previously (Iredale et al., (1998) supra).

Determination of Messenger RNA for TIMP-1 and GAPDH Using Taqman Real Time Quantitative PCR

Total RNA was extracted from snap frozen 6- and 12-week carbon tetrachloride treated rat livers at day 0 (peak fibrosis) and after 15 days of spontaneous recovery (Qiagen). The first strain cDNA synthesis was undertaken using random primers and the Moloney murine leukemia virus reverse transcriptase system (Promega). All primers and probes were designed using the Taqman Primer Express program, and real time Taqman PCR mRNA quantitation using the PerkinElmer Applied Biosystems 7700 Sequence Detection System. Primers and probe sequences of rat GAPDH used were as follows: sense, 5′-ggcctacatggcctccaa-3′ (SEQ ID NO:2); antisense, 5′-tctctcttgctctcagtatccttgc-3′ (SEQ ID NO:3); and probe, 5′-agaaaccctggaccacccagccc-3′ (SEQ ID NO:4). Rat TIMP-1 primers and probe sequences used were as follows: sense, 5′-agcctgtagctgtgccccaa-3′ (SEQ ID NO:5); antisense, 5′-aactcctcgctgcggttctg-3′ (SEQ ID NO:6); probe, 5′-agaggctctccatggctggggtgta-3′ (SEQ ID NO:7). 1 μl of first strand cDNA (10 ng of RNA), 0.3 μM primers, and 0.3 μM probe were used per 25-μl real time Taqman PCR. Taqman 2× Universal PCR Master Mix and 0.2 ml of optical reaction tube (PerkinElmer Applied Biosystems) were employed. The conditions of the reaction were as follows. Initial steps were 50° C. for 2 min and 95° C. for 10 min, followed with a denaturing step for 15 seconds at 95° C. and an annealing extension step at 60° C. for 1 min. Determination of the expression of the housekeeping gene, GAPDH, was employed, and all reactions were undertaken in triplicate. After detection of the threshold cycle for each mRNA in each sample, relative concentrations were calculated and normalized to GAPDH analyzed in parallel.

Enzyme-Linked Immunosorbent Assay for Fas and Fas Ligand

Human hepatic stellate cells were grown to confluence and exposed to BSA with and without TIMP-1. Cells and supernatants were harvested, and protein extracts were assayed for Fas and Fas ligand by commercial enzyme-linked immunosorbent assay following the manufacturer's instructions (Calbiochem). The quantities of Fas and Fas ligand were normalized to cell number by DNA quantification using the PicoGreen technique.

Results

TIMP-1 Inhibits Apoptosis Induced by Cycloheximide, Serum Deprivation, and Nerve Growth Factor

The experimental data provided in Example 1 indicated that it is possible to promote resolution of liver fibrosis by inducing hepatic stellate cell apoptosis. Here one of the factors which inhibits hepatic stellate cell apoptosis is investigated namely, the potential antiapoptotic effects of TIMP-1. This could therefore potentially provide a target for intervention in order to promote hepatic stellate cell apoptosis.

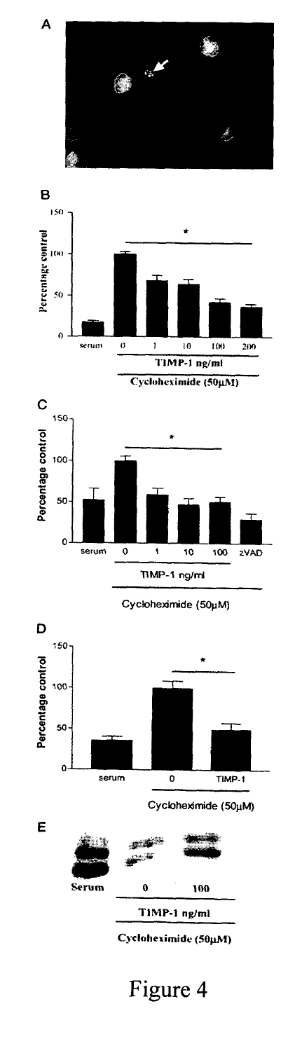

Apoptosis was assessed by acridine orange staining. FIG. 4A shows an example of an apoptotic hepatic stellate cell (arrow) induced by cycloheximide exposure for 4 hours. A normal cell lies adjacent to the apoptotic body. This technique was used to determine the percentage of apoptotic hepatic stellate cells following exposure to cyclohexamide in the presence or absence of TIMP-1. Hepatic stellate cells were exposed to 50 μM cyclohexamide and 0, 1, 10, 100 or 200 ng/ml of TIMP-1. Cells treated with serum alone were used as controls. The results obtained are shown in FIG. 4B. The results in FIG. 4B shows graphically the mean±S.E. expressed as percentage of control given the arbitrary value of 100%. * indicates p<0.001 for cycloheximide versus cycloheximide with 200 ng/ml TIMP-1 by Student's t test, n=5. The results show that TIMP-1 significantly reduces apoptosis of activated hepatic stellate cells induced by cycloheximide exposure in a dose-dependent manner over the concentration range 1-200 ng/ml. An identical effect with TIMP-1 was observed after a 24 hour incubation in serum-free conditions (data not shown). Bovine serum albumin, used as a carrier for the TIMP-1 had no antiapoptotic effect. Parallel experiments with human hepatic stellate cells treated with cycloheximide for 4 h or serum deprivation for 18 h demonstrated identical antiapoptotic effects for TIMP-1 (data not shown; n=4).

TIMP-1-Treated Hepatic Stellate Cells have Reduced Caspase-3 Activity Following Induction of Apoptosis by Cycloheximide

Caspase-3 is a central caspase in the proapoptotic cascade (Hengartner, Nature (2000) 407, 770-776) and can be used as an alternative assay to assess apoptosis. Hepatic stellate cells were cultured in 50 μM cycloheximide with TIMP-1 at a concentration of 0, 1, 10, or 100 ng/ml. Controls where cells were incubated with either the caspase 3 inhibitor benzyloxycarbonyl-Val-Ala-Asp-fluoromethylketone or serum alone were also performed. The results obtained are shown in FIG. 4C. Data are expressed as mean±S.E. and are presented as percentage of control given the arbitrary value of 100%. * indicates p<0.001; n=3. The results show that TIMP-1 treatment gives a dose-dependent reduction in caspase-3 activity over the concentration range 1-100 ng/ml tested. Although the mean caspase-3 activity of the cells treated with 10 ng/ml TIMP-1 was slightly lower than that treated with 100 ng/ml of TIMP-1, this was not statistically significant (p=0.67 by Student's t test). Therefore, this did not represent a reversal of the dose trend observed by acridine orange staining and counting.

The caspase-3 data and acridine orange morphological data did not correlate exactly with each other. For example, TIMP-1 at a concentration of 10 ng/ml caused a 50% reduction in caspase-3 activity, but only a 30% reduction in apoptotic morphology by acridine orange staining and counting. At the higher dose of 100 ng/ml, TIMP-1 appeared to reduce apoptosis by 50% measured by both techniques. These observed differences in dose response may be due to two factors. First, the precision of each assay is unlikely to be the same. Second, while the caspase-3 activity assay is accepted as a measure of apoptosis, it is at best only a measure of one out of the sixteen known caspase enzymes in what is clearly a complicated enzymatic cascade, which ends in the morphological changes that are characteristic of apoptosis.

To exclude a direct effect of TIMP-1 on caspase-3 activity, recombinant human caspase-3 (Calbiochem) was incubated with TIMP-1 in varying concentrations (285-2850 ng/ml) for 1 hour before caspase-3 substrate was added to the reaction. TIMP-1 did not reduce caspase-3 activity directly (data not shown).

TIMP-1-Treated Hepatic Stellate Cells have Reduced DNA Fragmentation Assessed by the TUNEL Technique Following Induction of Apoptosis by Cycloheximide

A further pathognomonic feature of apoptosis is the fragmentation of DNA into oligonucleosomal lengths (Evan et al., Cell (1992) 69: 119-128). Fragmented DNA can be identified by the TUNEL technique, which can therefore be used to further quantify the apoptotic response of hepatic stellate cells in the presence and absence of cycloheximide. To assess DNA fragmentation activated hepatic stellate cells were therefore induced to undergo apoptosis by cycloheximide treatment in the presence and absence of TIMP-1 and the number of TUNEL positive cells assessed. Activated hepatic stellate cells were cultured on glass chamber slides and exposed to cycloheximide for 18 h followed by treatment with either TIMP-1 or no TIMP-1. As a control cells which were treated with serum alone were anlaysed. The results obtained are presented graphically in FIG. 4C. Data are expressed as mean±S.E. and presented as percentage of control, which has been given the arbitrary value of 100. * indicates p<0.001; n=2. The results show that activated hepatic stellate cells treated with TIMP-1 demonstrate significantly reduced numbers of cells containing fragmented DNA as assessed by the TUNEL technique compared with controls treated without TIMP-1.

TIMP-1 Enhances Expression of Bcl-2 Protein

The protein Bcl-2 regulates the properties of cells to undergo apoptosis by interpolating into the mitochondria membrane (Hengartner, supra). Bcl-2 increases the resistance of cells to apoptosis. To define changes in the protein level of Bcl-2, extracts from hepatic stellate cells treated with cycloheximide for 18 hours, either in the presence and absence of 200 ng/ml TIMP-1, were analyzed by western blotting. Equal quantities (determined by protein concentration) of protein extracts from hepatic stellate cells exposed to: serum alone; cycloheximide alone; or cycloheximide with TIMP-1 protein (100 ng/ml) were assessed by blotting. The results obtained are shown in FIG. 4E. Relative to cells treated with cycloheximide alone, cells treated with both TIMP-1 and cycloheximide demonstrated enhanced levels of Bcl-2 protein expression, which approached the levels observed in hepatic stellate cells maintained in serum alone.

TIMP-1 Inhibits Apoptosis Induced by Nerve Growth Factor

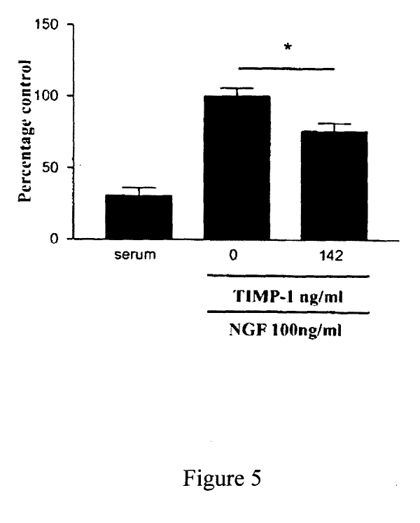

Hepatic stellate cells express low affinity nerve growth factor receptor (p75) and undergo apoptosis in response to nerve growth factor (NGF) stimulation. To determine whether TIMP-1 reduced NGF-induced apoptosis, NGF-activated hepatic stellate cells were exposed to NGF (100 ng/ml) in conditions of absolute serum deprivation with and without TIMP-1 (142.5 ng/ml). The results obtained are shown in FIG. 5. The data is expressed as mean±S.E. and presented relative to control given the arbitrary value of 100%. * indicates p<0.02 for NGF treated alone versus NGF with TIMP-1 treatment by Student's t test; n=3. As expected, NGF induced significantly more apoptosis in hepatic stellate cells than cells treated with BSA carrier alone (data not shown). Apoptosis induced by exposure to NGF in serum-free conditions was significantly inhibited by TIMP-1.

TIMP-1 is an Autocrine Survival Factor for HSC

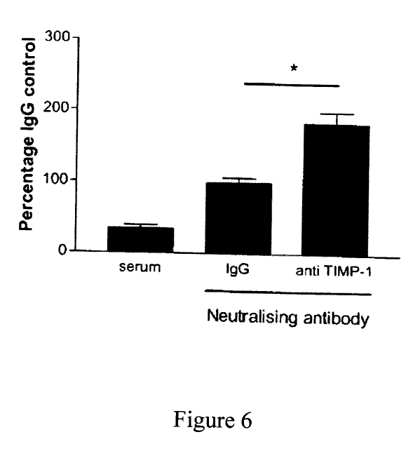

TIMP-1 is major synthetic product of activated hepatic stellate cells. Therefore, TIMP-1 is potentially an autocrine survival factor for hepatic stellate cells. To determine the effect of neutralizing hepatic stellate cell derived TIMP-1, hepatic stellate cells were incubated with azide free polyclonal neutralizing antibodies to TIMP-1 for 18 h in 5% bovine serum albumin and compared with a nonimmune IgG control antibody as described under Materials and Methods. All antibodies were in azide-free buffer. Apoptosis was quantified by the acridine orange technique. The results obtained are shown in FIG. 6. Data is expressed as mean±S.E. and presented relative to control, which has been given the arbitrary value of 100%. * indicates p<0.0001 by Student's t test for hepatic stellate cells treated with neutralizing antibodies for TIMP-1 relative to nonimmune IgG control (n=3). The results show that neutralizing TIMP-1 antibody significantly increases apoptosis of activated hepatic stellate cells compared with exposure to the nonimmune IgG control suggesting that TIMP-1 acts as a survival factor in an autocrine manner for activated hepatic stellate cells.

The Antiapoptotic Effect of TIMP-1 for Hepatic Stellate Cells is Mediated Via MMP Inhibition

To determine whether the antiapoptotic activity of TIMP-1 might be mediated via MMP inhibition, further experiments were performed using a mutated nonfunctional TIMP-1 (T2G) in which all other domains were conserved (Meng et al., J. Biol. Chem. (1999) 274: 10184-10189).

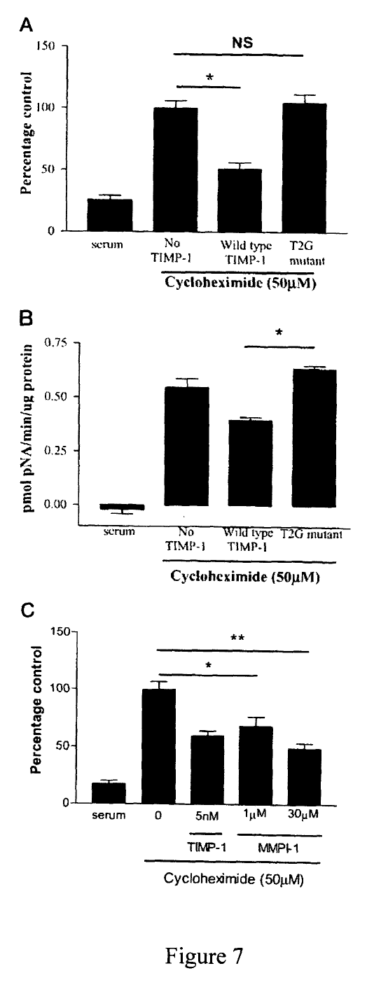

Hepatic stellate cells were exposed to cycloheximide in the presence or absence of wild type or T2G mutant TIMP-1. The percentage of apoptotic cells was assessed by acridine orange staining . The results obtained are shown in FIG. 7A. Data are expressed as mean±S.E. and presented as a percentage of control, which has been given the arbitrary value of 100%. *, p<0.01 by Student's t test. NS, not significant by Student's t test; n=3. The T2G mutant N-TIMP-1 had no inhibitory effect on rat or human hepatic stellate cell apoptosis induced by cycloheximide, whereas the wild type N-TIMP-1 protein at an identical concentration (142.5 ng/ml) significantly inhibited apoptosis. Thus it appears that MMP inhibitory activity is necessary for the antiapoptotic affect of TIMP-1.

The level of casapase 3 activity was also assessed in hepatic stellate cells treated with cyclohexamide and either wild type TIMP-1 or the T2G mutant N-TIMP-1. The results obtained are shown in FIG. 7B. Data are presented as mean±S.E.; p<0.01; n=3. The wild type TIMP-1 significantly reduced caspase-3 activity relative to the T2G mutant. The results show that while the wild type TIMP-1 reduced caspase-3 activity in hepatic stellate cells treated with cycloheximide, no effect was observed with the T2G nonfunctional mutant. Again this suggests that the inhibition of apoptosis by TIMP-1 was MMP-dependent.

A series of experiments were then performed using a synthetic matrix metalloproteinase inhibitor (MMPI-1;Calbiochem). Cells were treated with cycloheximide and either TIMP-1 (at 142.5 ng/ml equivalent to 5 nM/L) or MMPI-1 (at either 1 or 30 μM concentration). Controls were performed using cells treated with cycloheximide alone or serum alone. The percentage of apoptotic cells was then assessed by acridine orange staining and cell counting. The results obtained are shown in FIG. 7C. Data are expressed as mean±S.E. and presented relative to control, which has been given the arbitrary value of 100%. *,p<0.001; * *,p<0.0001 by Student's t test; n=3. As previously described, TIMP-1 inhibited apoptosis induced by cycloheximide exposure. The synthetic matrix metalloproteinase inhibitor MMPI-1 also demonstrated a dose-dependent protective effect at a concentration of 1-30 μM. The concentration of inhibitor used was calculated to provide a level of MMP inhibition comparable with 142.5 ng/ml recombinant TIMP-1 on the basis of the published Kᵢ for the inhibitor and the recombinant TIMP-1. The results suggest that the antiapoptotic effect in hepatic stellate cells could be brought about by matrix metalloproteinase inhibition alone.

Effect of TIMP-1 on FAS/APO-1/CD95 and Fas Ligand

Experiments were next performed to determine whether TIMP-1 regulated Fas ligand cleavage in human hepatic stellate cells. Hepatic stellate cells were incubated for 18 hours in conditions of absolute serum deprivation with BSA or BSA with TIMP-1 (142.5 ng/ml). The cells were extracted, and supernatants were collected. After normalizing for cell number (by DNA concentration using the PicoGreen technique), these extracts were analyzed by enzyme-linked immunosorbent assay for Fas and Fas ligand as described under Materials and Methods. TIMP-1 treatment of human hepatic stellate cells had no effect on cellular Fas or Fas ligand protein levels compared with control cells treated with BSA alone. Supernatant Fas and Fas ligand protein levels were undetectable in all experimental conditions (data not shown, n 3). Thus it appears that TIMP-1 does not mediate its anti-apoptotic effects via regulation of Fas cleavage.

TIMP-1 has No Effect on Hepatic Stellate Cell Proliferation

As previous studies have demonstrated a potential proproliferative effect for TIMP-1, this was analyzed in activated hepatic stellate cells. TIMP-1 at concentrations of 1-100 ng/ml had no effect on proliferation of rat hepatic stellate cells (n=4) over a 24 hour incubation period compared with bovine serum albumin carrier used as a negative control (data not shown).

Persistence of TIMP-1 Expression is Accompanied by Persistence of Activated Hepatic Stellate Cells and Decreased Resolution of Liver Fibrosis

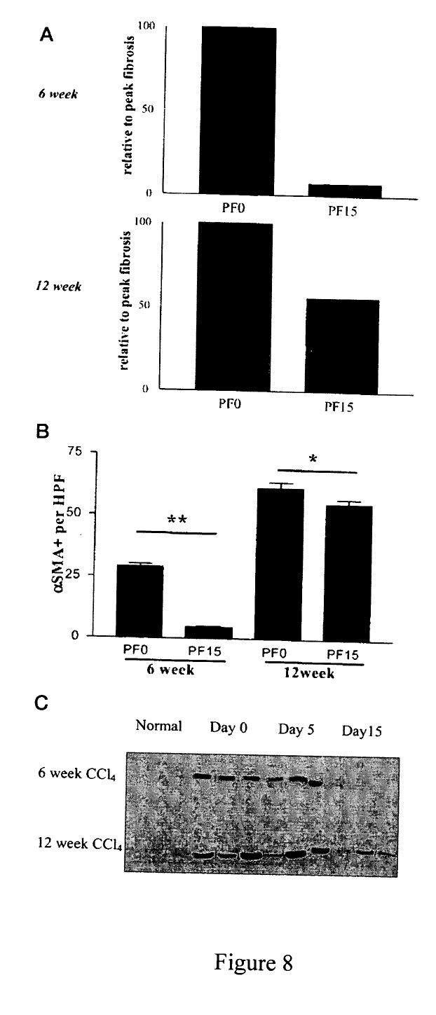

TIMP-1 levels fall during spontaneous recovery of experimental fibrosis following four weeks of carbon tetrachloride intoxication (Iredale et al -1998-supra). To determine whether TIMP-1 mRNA remained elevated in liver cirrhosis, a further model of experimental fibrosis was undertaken. Rats injured with carbon tetrachloride as described under Materials and Methods were harvested after 12 and 6 weeks of intoxication and after a further 5 and 15 days of spontaneous recovery for each model. TIMP-1 mRNA expression was determined by Taqman quantitative PCR in total liver RNA and the results obtained are shown in FIG. 8A (PF0, peak fibrosis, immediately after the final injection of carbon tetrachloride; PF15, after 15 days of spontaneous recovery). Data is presented as mean change relative to peak fibrosis, which has been given the arbitrary value of 100 for each data set. All values have been normalized for GAPDH expression determined in parallel. The results show that after 6 weeks of treatment with carbon tetrachloride, a 13-fold decrease in TIMP-1 expression occurs during the first 2 weeks of spontaneous recovery (compare PF0 and PF15; 6 weeks of CCl₄- the top panel of FIG. 8A). In contrast, there is only a two fold fall in TIMP-1 mRNA during the first 15 days of recovery in the 12 week injured rat liver (compare PF0 and PF15; 12 weeks of CCl₄—the bottom panel of FIG. 8A).

To determine whether the persistence of TIMP-1 expression after 12 weeks of carbon tetrachloride correlated with persistence of activated hepatic stellate cells and a failure of matrix degradation, immunohistochemistry for smooth muscle actin and histological analysis were undertaken on the same livers.

The numbers of smooth muscle actin (SMA)-positive HSC were quantified in section form after 6 and 12 weeks of carbon tetrachloride intoxication and after 15 days of spontaneous recovery as described under Materials and Methods. The results obtained are shown in FIG. 8B (data presented are mean±S.E.; n=4 for each experimental group at each time point; **, p<0.0001; *, p<0.03). During the first two weeks of spontaneous recovery from rat liver fibrosis, there is minimal change in the number of smooth muscle actin-positive hepatic stellate cells in the 12-week injured liver (compare PF0 and PF15; 12 weeks), whereas in the liver injured for 6 weeks, there is a dramatic decrease in the number of smooth muscle actin-positive staining cells (compare PF0 and PF15; 6 weeks). In the 6-week model during 15 days of recovery there was also a 50% drop in liver hydroxyproline content to a level identical to that seen in untreated control liver. In contrast, the 12-week model showed increased levels of hydroxyproline of 150% of normal liver at peak fibrosis, which did not significantly change over the 15 days of spontaneous recovery.

The results of the Western blotting experiments are shown in FIG. 8C (Normal, untreated liver control; Day 0, immediately after the final injection of carbon tetrachloride; Day 5 and Day 15, after 5 and 15 days of spontaneous recovery, respectively; n=3 for each time point). The western blotting of whole liver homogenate for smooth muscle actin demonstrates reduction in levels of liver smooth muscle actin protein over the first 15 days of recovery after 6 weeks of carbon tetrachloride intoxication (compare Day 0 and Day 15; 6 weeks of CCl₄). In contrast, levels of smooth muscle actin protein remain elevated in whole liver extracts from the animals injured with carbon tetrachloride for 12 weeks even after 15 days of spontaneous recovery (compare Day 0 and Day 15; 12 weeks of CCl₄). In both models, the liver smooth muscle actin protein level is increased at peak fibrosis (Day 0) relative to normal livers. Thus, both immunostaining of sections for smooth muscle actin with cell counting and western analysis of liver homogenates for smooth muscle actin demonstrated that there was only a slight decrease in smooth muscle actin-positive activated hepatic stellate cells during recovery with significant numbers of smooth muscle actin-positive activated hepatic stellate cells present in the 15-day recovery livers after 12 weeks of carbon tetrachloride.

Histological analysis by Sirius Red stain of rat livers harvested after 6 and 12 weeks of carbon tetrachloride intoxication twice weekly as described under Materials and Methods was carried out. Livers were harvested at peak fibrosis (PF0) following 12 and 6 weeks of treatment and after a further 15 days of spontaneous recovery. In the 12-week model, there was more substantial fibrosis (indeed, cirrhosis is present) compared with the 6 weeks of injury. Furthermore, there was evidence of only modest matrix remodeling during the 15 days of spontaneous recovery in the 12-week model. In the 6-week model, established septal fibrosis was present, which demonstrates evidence of remodeling over 15 days.

To determine whether the observed changes in TIMP-1 mRNA expression were associated with MMP inhibition, analysis of collagenase activity in whole liver homogenate was undertaken. This demonstrated that after 12 weeks of carbon tetrachloride, at no time point (days 0 or 5 or 15 days of recovery) was activity above that seen in normal untreated liver (collagenase activities expressed as percentage of normal liver ±S.E. were as follows: day 0, 70±1.9%; day 5, 60±3.3%; day 15, 55±3.7%). In contrast, after 6 weeks of carbon tetrachloride, collagenase activity in the liver homogenates demonstrated an increase, peaking at 5 days of recovery (collagenase activities expressed as percentage of normal liver ±S.E. at each time point were as follows: day 0, 70±1.9%; day 5, 147±3.3%; day 15, 107±1.6%). Taken together the results obtained demonstrate a strong correlation between persistence of activated hepatic stellate cells following fibrotic injury and TIMP-1 expression and a failure of matrix degradation with persistent inhibition of collagenase activity.

Discussion

The results obtained here demonstrate that TIMP-1 promotes survival of activated hepatic stellate cells and provide cogent evidence that this effect is specifically mediated via inhibition of matrix matalloprotease (MMP) activity. Moreover, this functional data has been combined with evidence for a correlation of TIMP-1 expression and survival of activated hepatic stellate cells in vivo after withdrawal of a toxic injury.

During recovery from liver fibrosis in the rat carbon tetrachloride and bile duct ligation model of fibrosis, there is a diminution of hepatic stellate cell number mediated by apoptosis. At the same time, there is a reduced expression of TIMP-1. These studies and the data reported here address a crucial question to our understanding of liver fibrosis: What determines whether a fibrotic liver injury recovers or fails to recover?

In recovery there is a net reduction in activated hepatic stellate cells and fibrotic matrix, whereas in progressive fibrosis, the activated hepatic stellate cells and neomatrix remain. Identification of factors promoting the survival of activated hepatic stellate cells is therefore essential to understanding the pathogenesis of fibrosis and also for devising ways to promote resolution of fibrosis. TIMP-1 is an important potential candidate mediating hepatic stellate cell survival.

An exhaustive series of experiments have been undertaken here using the established and robust model of activated stellate cells in tissue culture to analyze the influence of TIMP-1 on hepatic stellate cell apoptosis induced by a variety of stimuli. The results obtained in tissue culture indicate that TIMP-1 has a direct, consistent, significant, and concentration-dependent antiapoptotic effect on both human and rat hepatic stellate cells. It has been shown, using a series of complementary quantitative techniques, that TIMP-1 reduces apoptosis induced by serum deprivation, cycloheximide exposure, and nerve growth factor stimulation and that this effect is shared by both rat and human hepatic stellate cells, suggesting that it is a biologically important phenomenon. Furthermore, despite the variety of means of induction of apoptosis, the antiapoptotic effect of TIMP-1 is remarkably consistent. TIMP-1 had no proproliferative effect on activated hepatic stellate cells. From a biological view, it would seem undesirable for a protein to both inhibit apoptosis and promote proliferation in the same cell type, since expression of such a protein would be potentially carcinogenic.

Further experiments were performed to define the mechanism whereby TIMP-1 inhibits apoptosis. This was approached in two ways, by using the published Kᵢ values of the reagents employed to use comparable inhibitory concentrations of synthetic inhibitor to recombinant TIMP-1 and by using the T2G mutant N-TIMP-1. Studies with the synthetic MMP inhibitor, MMPI-1, suggest that MMP inhibition is likely to be the mechanism mediating survival of hepatic stellate cells. Using the T2G mutant N-TIMP-1, it was demonstrated directly that inhibition of apoptosis of hepatic stellate cells by TIMP-1 is in fact mediated via its effects on MMP activity. The T2G mutant N-TIMP-1 protein differs from the wild type protein by only a single amino acid substitution (threonine to glycine at amino acid position 2), which reduces the inhibition constant of TIMP-1 for MMP-1 and MMP-3 by a factor of over 1000. Moreover, the secondary structure of this mutant protein is not significantly different from the wild type. This makes it the best available reagent available to address the issue of MMP dependence in protection from apoptosis. At the dose of TIMP-1 used in these experiments (142.5 ng/ml), the mutant TIMP-1 would have effectively no MMP inhibitory activity, whereas the wild type TIMP-1 would be expected to significantly reduce MMP activity.

The potential mechanisms through which apoptosis may be regulated by TIMP-1 are legion and may involve more than one MMP. A major candidate mechanism through which TIMPs mediate survival is by preventing matrix degradation. Hepatic stellate cells may gain direct signals from matrix. Moreover, matrix contains numerous matrix-bound cytokines that may have antiproliferative and/or proapoptotic effects on local cell populations (e.g. transforming growth factor) that may be liberated by matrix degradation. In the context of the liver fibrosis recovery model (Iredale et al—1998—supra), during the degradation of fibrotic tissue, release of matrix-bound cytokines may also be important in determining the pattern of recovery and apoptosis of activated hepatic stellate cells. If TIMP-1 reduces apoptosis via preventing matrix degradation, it may do this by preventing MMP degradation of some key targets. First, release of matrix-bound proapoptotic factors would be prevented. Second, intact matrix may provide direct cell survival signals and present matrix-bound survival signals in a spatially effective manner. TIMP would preserve such signals. In support of this hypothesis, it has been found that a mutant collagen, resistant to collagenase digestion, will promote hepatic stellate cell survival in models of fibrosis. Moreover, the in vivo studies provided here are compatible with TIMP-1 promoting hepatic stellate cell survival through MMP inhibition and protection of the fibrotic matrix.

Results from our previous 4-week model of rat liver fibrosis and the 6-week carbon tetrachloride model reported here indicate that spontaneous recovery is associated with a decrease in the number of smooth muscle actin-positive cells. Furthermore, this is associated with a large decrease in TIMP-1 mRNA expression and an increase in collagenase activity that parallels the changes in smooth muscle actin. In contrast, after 12 weeks of carbon tetrachloride cirrhosis results, and there is only minimal evidence of matrix remodeling, no increase in collagenase activity, and a persistence of activated hepatic stellate cells. TIMP-1 expression actually decreased modestly over 15 days of spontaneous recovery in the 12-week model. This result is to be expected, since TIMP-1 is expressed by inflammatory cells and in the acute response to injury (Iredale et al., Hepatology (1996) 24: 176-184). Nevertheless, after 15 days of recovery in the 12-week carbon tetrachloride model, significant expression of TIMP-1 remains. This in vivo evidence strongly suggests that TIMP-1-mediated MMP inhibition is a unifying mechanism promoting survival of activated hepatic stellate cells and protecting the fibrotic matrix from degradation.

There are further mechanisms by which MMP inhibition may mediate survival in vivo. It is known that many cell surface proteins can be cleaved, provided their appropriate “sheddase” is present and active. In cases where MMPs mediate shedding of receptors (e.g. tumor necrosis factor receptor), TIMPs may indirectly regulate cell behavior. Recently, TIMP-3 has been demonstrated to induce apoptosis in human colonic carcinoma cells by stabilizing tumor necrosis factor receptors on the cell surface (Smith et al., Cytokine (1997) 9: 770-780. Endothelial cells have been demonstrated to shed receptors for tumor necrosis factor following induction of apoptosis, which may be a mechanism to limit inflammation in response to apoptotic cell death (Madge et al., J. Biol. Chem., (1999) 274, 13643-13649). A further MMP-dependent cell surface protein system regulating apoptosis is the Fas/Fas ligand system. Hepatic stellate cells are known to express Fas and Fas ligand on their cell surface (Saile et al., Am. J. Pathol., (1997) 151, 1265-1272 and Gong et al., Hepatology (1998) 28: 492-502). TIMP-1 did not have any effect on cellular Fas or Fas ligand protein levels in activated human hepatic stellate cells. It is also possible that TIMP-1 might inhibit apoptosis by preventing the shedding of a prosurvival receptor (e.g. insulin-like growth factor-1 receptor), which is known to prevent apoptosis in activated hepatic stellate cells and related cells (Issa et al., Gut (2001) 48, S48-S57 and Baker et al., J. Clin. Invest. (1994) 94: 2105-2116). A further MMP-cleaved cell surface receptor that regulates cell survival is cadherin. The cadherin and catenin pathway is known to impact on cellular Bcl-2 levels and thus the inherent tendency for a given cell to undergo apoptosis (Herren et al., Mol. Biol. Cell (1998) 9: 1589-1601).

The data described in this study provide strong evidence that TIMP-1 is mechanistically important in promoting fibrosis by inhibiting the apoptosis of activated hepatic stellate cells by a process that is also MMP-dependent. This observation highlights TIMP-1 as an important therapeutic target in the treatment of liver cirrhosis.

### Example 3

### Antagonists of 5HT₂ Receptors can be Used to Stimulate Hepatic Stellate Cell Apoptosis

Total RNA was extracted from freshly isolated rat hepatic stellate cells, hepatocytes and 10 day cultures activated hepatic stellate cells from which cDNA was reverse transcribed using random hexamers as the primers in all cases. The cDNAs were then primed with oligonucleotides specific to the rat 5-HT_{2A}, 5-HT_{2B} and 5-HT_{2C} receptors and amplified using PCR for up to 40 cycles before agarose resolution.

PCR demonstrated that the mRNA for the 5-HT_{2A} receptor was present in both freshly isolated and 10 day culture activated hepatic stellate cells as well as freshly isolated hepatocytes. The mRNA for the 5-HT_{2B} receptor was only found in 10 day culture activated hepatic stellate cells, whereas the mRNA for the 5-HT_{2C} receptor was absent from all cells investigated. Western blots performed with rabbit anti-5-HT_{2A} polyclonal antibodies also indicated that 5-HT_{2A} receptor protein was present in 10 day culture activated hepatic stellate cells and to a lesser extent in freshly isolated hepatic stellate cells. 10 day culture activated hepatic stellate cells were treated with a range of 5-HT₂ antagonists (including Spiperone HCl, Methiothepin Maleate and LY 53,857) at various concentrations and time periods and nuclear morphology was assessed using Acridine Orange (1 μg/ml) staining as described in Examples 1 and 2. LY 53,857 was found to cause maximal nuclear condensation (approaching 100%) when cells were treated for 24 hours at 100 M. Methiothepin maleate was found to cause maximal nuclear condensation (100%) when cells were treated for 3 hours at 10-100 μM. Spiperone was found to cause maximal nuclear condensation (80%) when cells were treated for 24 hours at 100 μM. Caspase 3 activity (as defined by pNA liberation) was subsequently determined at the predetermined optimal times and doses as described in Examples 1 and 2. 10 day culture activated hepatic stellate cells were also treated with the antagonist together with the 5-HT₂ agonist serotonin (5-hydroxytryptamine 100 μM) to see if any modification of the caspase 3 specific activity could be achieved by direct receptor binding site competition. Treatment of cells with LY 53,857 at 100 μM for 24 hours resulted in a specific caspase 3 activity of 1 pmol pNA liberated/hour/μg protein which was not significantly reduced by the presence of serotonin. Treatment of cells with Methiothepin maleate at 10 μM for 3 hours resulted in a specific caspase 3 activity of 0.9 pmol pNA liberated/hour/μg protein which was not significantly reduced by the presence of serotonin. Treatment of cells with Spiperone at 100 μM for 24 hours resulted in a specific caspase 3 activity of 6.5 pmol pNA liberated/hour/μg protein which was significantly reduced (P<0.05) by the presence of serotonin to 4 pmol pNA liberated/hr/μg protein.

In conclusion rat hepatic stellate cells express 5-HT₂ receptors of which the 5-HT_{2B} subtype is absent on hepatocytes. Moreover, treatment of hepatic stellate cells with antagonists against these receptors will promote elevated rates of hepatic stellate cell apoptosis and hence can be used to treat liver disease and in particular liver fibrosis.

### Example 4

### Inhibition of NFκB Activity and Induction of Hepatic Stellate Cell Apoptosis by Sulfasalazine

The anti-inflammatory, immuno-suppressive drug Sulfasalazine, a known IKK inhibitor (Weber et al., Gasteroenterology, (2000) 119, 1209-18) was used to determine the role of NFκB in regulating stellate cell apoptosis.

Electromobility Shift Assay analysis revealed that treatment of day 7 hepatic stellate cells with 0.5, 1 and 2 mM Sulfasalazine for 24 hours dose dependently inhibited NFκB DNA binding activity, but not that of the transcription factors CBF1 and upstream TIMP1 binding element (UTE1), compared to control cells.

Sulfasalazine treatment for 24 hours repressed the activity of three NFκB responsive gene promoters (PGJ21 (4×NFκB), IL6 and IκBα), but not a control 7×AP1 promoter which is not NFκB responsive, compared to untreated cells.

Treatment of activated hepatic stellate cells with Sulfasalazine (at a concentration of 0.5, 1 or 2 mM) for 24 hours induced a dose-dependent increase in apoptosis visualised by both Acridine Orange staining (30%, 45% and 55% respectively). Sulfasalazine (at a concentration of 0.5, 1 or 2 mM) also increased caspase 3 activity in a dose-dependent manner.

### Example 5

### Effect of Sulfasalazine on Experimentally Induced Fibrosis

Following our finding that sulfasalazine will promote the apoptosis of hepatic stellate cells we have gone on to test the ability of the compound to accelerate recovery from experimentally induced fibrosis in rats.

Adult male Sprague Dawley rats were treated twice weekly for six weeks by intraperitoneal injection of the hepatotoxin carbon tetrachloride and then allowed to recover for 24 hours prior to treatment with either vehicle control or sulfasalazine. Animals were then left to recover for either a further 16 or 72 hours. At 16 hours recovery we observed a large reduction in numbers of activated hepatic stellate cells in sulfasalazine treated livers compared to control livers, however no significant difference in collagen deposition (fibrosis) was observed. At 72 hours recovery there was significantly less collagen in sulfasalazine treated livers which was confirmed independently by an expert pathologist who graded these livers with a fibrosis score of 1.5 versus a score of 3 for the control livers (a 1-4 scoring system is used clinically with 1 being no fibrosis and 4 being cirrhosis).

These data indicate that sulfasalzine treatment will promote the rapid removal of collagen producing activated hepatic stellate cells from diseased liver and will then enable subsequent degradation of collagen so as to promote normal tissue repair.

1. A method of treating liver disease in a subject, the method comprising administering to said subject an effective amount of an inducer of hepatic stellate cell apoptosis or of an agent capable of giving rise to an inducer of hepatic stellate cell apoptosis, wherein said inducer or agent:
(a) is selectively delivered to hepatic stellate cells in the liver of the subject;
(b) selectively induces, or gives rise to a selective inducer, of hepatic stellate cell apoptosis in the liver of the subject; and/or
(c) generates an inducer of apoptosis specifically in hepatic stellate cells.

2. A method according to claim 1, wherein the number of hepatic stellate cells induced to undergo apoptosis in the liver of the subject is at least ten times greater than the number of hepatocytes induced to undergo apoptosis.

3. A method according to claim 1, wherein the inducer of apoptosis administered to the subject, or which the agent gives rise to, induces hepatic stellate apoptosis in the liver of the subject, but does not induce apoptosis of other cell types in the liver of the subject.

4. A method according to claim 1, wherein the inducer of apoptosis administered or generated can only induce hepatic stellate cell apoptosis in the liver of the subject and is incapable of inducing apoptosis of any other cell type in the body of the subject.

5. A method according to claim 1, wherein the inducer or agent administered to the subject specifically binds to a molecule which is found on the surface of the hepatic stellate cells of the subject, but not on the surface of other liver cell types.

6. A method according to claim 5, wherein the inducer or agent administered to the subject binds to a molecule which is present on the surface of the hepatic stellate cells of the subject, but which is not present on the surface of other cell types in the body of the subject.

7. A method according to claim 5, wherein the molecule bound by the inducer is a cell surface receptor and the binding of the receptor triggers apoptosis.

8. A method according to claim 5, where the binding of the molecule by the receptor results in the internalization of the inducer or agent into the hepatic stellate cell.

9. A method according to claim 1, wherein the inducer administered or generated is an antagonist of a 5HT₂ receptor.

10. A method according to claim 9, wherein the inducer is an antagonist of the 5HT_{2B} receptor subtype.

11. A method according to claim 1, wherein the inducer or agent is delivered to the hepatic stellate cells of the subject using a liposome or a virus.

12. A method according to claim 1, wherein the agent administered to the subject comprises a nucleic acid construct which:
encodes a polypeptide inducer of hepatic stellate cell apoptosis;
can be transcribed to give rise to an RNA molecule which can induce hepatic stellate cell apoptosis; and/or
encodes a polypeptide whose expression results in the generation of an inducer of apoptosis.

13. A method according to claim 12, wherein the nucleic acid in the agent administered to the subject which encodes the polypeptide or which can be transcribed to give an RNA inducer is operably linked to a hepatic stellate specific promoter and hence is only expressed in the hepatic stellate cells of the subject.

14. A method according to claim 12, wherein the nucleic acid in the agent administered to the subject comprises a nucleic acid region capable of expressing an antisense nucleic acid or a siRNA molecule which induces hepatic stellate cell apoptosis.

15. A method according to claim 1, wherein the inducer or agent administered to the subject is specifically delivered to hepatic stellate cells using a receptor which occurs on the surface of hepatic stellate cells of the liver of the subject, but not other cell types in the liver.

16. A method according to claim 15, wherein the inducer or agent administered to the subject, is delivered using, or comprises, a liposome or virus which carries a molecule capable of binding the receptor occurring on the surface of the hepatic stellate cells of the subject and internalizing the inducer or agent into the cell.

17. A method according to claim 1, wherein the inducer of hepatic stellate cell apoptosis administered to the subject is selected from the group consisting of gliotoxin or a derivative of gliotoxin capable of inducing hepatic stellate cell apoptosis.

18. A method according to claim 17, wherein the gliotoxin, or derivative, is administered to the subject in an amount of from 0.1 to 25 mg per kg bodyweight of the subject.

19. A method according to claim 1, wherein the inducer of hepatic stellate cell apoptosis administered to the subject, or generated, is selected from the group consisting of nerve growth factor, a derivative of nerve growth factor or an antagonist of the p75 receptor.

20. A method according to claim 19, wherein the antagonist of the p75 receptor is spiperone or a derivative thereof.

21. A method according to claim 1, wherein the inducer administered to the subject, or generated, inhibits the interaction of a tissue inhibitor of a matrixmetalloprotease (TIMP) with a matrixmetalloprotease.

22. A method according to claim 21, wherein the inducer administered to the subject, or generated, inhibits the interaction of TIMP-1 with an MMP.

23. A method according to claim 1, wherein the inducer administered or generated is sulfasalazine or a derivative thereof capable of inducing hepatic stellate cell apoptosis.

24. A method according to claim 1, wherein the inducer or agent is admininistered to the subject in the form of an implant comprising the inducer or agent.

25. A method according to claim 24, wherein the implant is inserted into the liver of the subject.

26. A method according to claim 1, wherein the subject to be treated has liver cirrhosis.

27. A method according to claim 1, wherein the subject has a condition selected from the group consisting of fibrosis caused by a pathogen, fibrosis caused by an autoimmune condition, fibrosis due to exposure to a drug, fibrosis caused by exposure to a chemical, fibrosis caused by consumption of alcohol, fibrosis caused by an inherited condition and primary biliary cirrhosis.

28. A kit comprising:
a selective inducer of hepatic stellate cell apoptosis or an agent capable of giving rise to a selective inducer of hepatic stellate cell apoptosis in vivo; and
instructions describing how to administer the inducer or agent to a subject suffering from liver disease.

29. A kit comprising:
an inducer of hepatic stellate cell apoptosis or an agent capable of giving rise to an inducer of hepatic stellate cell apoptosis in vivo;
instructions describing how to selectively deliver the inducer or agent to the hepatic stellate cells of a subject suffering from liver disease.
